(19) United States
(12) Patent Application Publication
(10) Pub. No.: US 2014/0294788 A1
(43) Pub. Date: Oct. 2, 2014

(54) METHODS FOR ENHANCING MUSCLE PROTEIN SYNTHESIS FOLLOWING CONCURRENT TRAINING

(71) Applicant: Nestec S.A., Vevey (CH)

(72) Inventors: David Mark BAILEY, Vaud (CH); Eric Scott ZALTAS, Montclair, NJ (US); Daniel Ryan MOORE, Waterdown (CA); Trent STELLINGWERFF, Victoria (CA)

(73) Assignee: Nestec S.A., Vevey (CH)

(21) Appl. No.: 14/220,548

(22) Filed: Mar. 20, 2014

Related U.S. Application Data
(60) Provisional application No. 61/805,222, filed on Mar. 26, 2013.
(60) Provisional application No. 61/816,159, filed on Apr. 26, 2013.

(51) Int. Cl. A61K 38/02; A61K 45/06

(52) U.S. Cl. CPC A61K 38/02; A61K 45/06
USPC 424/93.41; 514/1.1; 424/93.44; 424/93.45; 424/93.43; 424/93.42

(57) ABSTRACT

The present disclosure provides methods for enhancing muscle protein synthesis following physical exertion. In a general embodiment, a method for enhancing muscle protein synthesis following physical exertion is provided and includes administering to an individual a composition comprising from about 15 to about 35 g protein immediately following concurrent training. Programs for enhancing muscle adaptation resulting from concurrent training are also provided. The programs include providing a composition comprising from about 15 to about 35 g protein; and providing guidelines for consumption including a recommendation of the amount of the composition to consume immediately following concurrent training.

### BACKGROUND

The present disclosure relates generally to health and fitness. More specifically, the present disclosure relates to methods for enhancing muscle protein synthesis following concurrent training.

Physical exercise alters the activity of proteins involved in ‘turning on’ protein synthesis (i.e., signaling molecules), determining which muscle proteins are synthesized as well as when this synthesis occurs. Similar to the molecular responses, the changes in protein synthesis after a single bout of exercise are largely specific to the exercise task, such as an increased synthesis of proteins involved in enhanced strength (i.e., myofibrillar) with resistance exercise and an increase of proteins involved with energy supply (i.e., mitochondrial) with aerobic exercise. For repeated physical exercise, these changes in signaling molecule activity and muscle protein synthesis summate over a period of weeks and months (i.e., with training) into physiological adaptations that make an athlete better at a specific exercise task/event.

There is limited information regarding specifically what and how nutrition can support and optimize these training adaptations. Further, many exercise tasks have a resistive component followed by an aerobic component and therefore it is relatively unknown precisely what changes occur in the molecular signaling pathways as well as the synthesis of different muscle proteins.

Exercise and nutrition (specifically protein ingestion) are potent stimulators of muscle protein synthesis with the combination of the two being synergistic. The stimulation of muscle protein synthesis has been shown to be protein fraction specific and dependent on the specific exercise stimulus. For example, resistance exercise typically stimulates increases in the synthesis of the mitochondrial protein fraction, including myofibrillar protein fractions, whereas aerobic exercise preferentially increases the mitochondrial protein fraction. However, it is not uncommon for sports athletes to perform both resistance and endurance exercise when training for a specific sports performance. This combination of exercise is commonly referred to as concurrent training and has efficacy as the specific adaptations from each mode are beneficial irrespective of the endurance or resistance focus of the sports performance targeted. Therefore, there exists a need to determine the potential impact of protein ingestion on the adaptations from concurrent training

### SUMMARY

In the present disclosure, methods for enhancing muscle protein synthesis are provided. In an embodiment, methods for enhancing muscle protein synthesis following physical exertion are provided. The method includes administering to an individual a composition comprising from about 15 to about 35 g protein immediately following concurrent training.

In another embodiment, methods for enhancing mitochondrial protein synthesis are provided. The methods include administering to an individual a composition comprising from about 15 to about 35 g protein immediately following concurrent training.

In another embodiment, methods for enhancing myofibrillar protein synthesis are provided. The methods include administering to an individual a composition comprising from about 15 to about 35 g protein immediately following concurrent training.

In yet another embodiment, programs for enhancing muscle adaptation resulting from concurrent training are provided. The programs are aimed at providing nutrition and guidance on training to an athlete to improve the muscle protein synthesis. The programs include providing a composition including from about 15 to about 35 g protein; and providing guidelines for consumption including a recommendation of the amount of the composition to consume immediately following concurrent training.

In an embodiment, the composition includes from about 20 g to about 30 g protein, or about 25 g protein.

In an embodiment, the composition includes essential amino acids selected from the group consisting of phenylalanine, valine, threonine, tryptophan, isoleucine, methionine, leucine, lysine, histidine, or combinations thereof.

In an embodiment, the composition is enriched with L-[ring-13C6] phenylalanine in an amount up to about 10% by weight of the composition, or up to about 5% by weight of the composition.

In an embodiment, the protein synthesis enhanced is mitochondrial protein synthesis.

In an embodiment, the protein synthesis enhanced is myofibrillar protein synthesis.

The composition may be in a form selected from the group consisting of a solid, a gel, a liquid, a ready-to-mix powder, or combinations thereof. In an embodiment, the composition is a liquid.

In an embodiment, a serving size of the composition is about 500 mL.

In an embodiment, the composition is administered from about 0 to about 30 minutes after concurrent training, or from about 2 to about 15 minutes after concurrent training, or from about 5 to about 10 minutes after concurrent training, or within about 5 minutes after concurrent training.

In an embodiment, the protein is selected from the group consisting of dairy based proteins, plant based proteins, animal based proteins, artificial proteins, or combinations thereof. The dairy based proteins may be selected from the group consisting of casein, caseinates, casein hydrolysate, whey, whey hydrolysates, whey concentrates, whey isolates, milk protein concentrate, milk protein isolate, or combinations thereof. The plant based proteins may be selected from the group consisting of soy protein, pea protein, canola protein, wheat and fractionated wheat proteins, corn proteins, zein proteins, rice proteins, oat proteins, potato proteins, peanut proteins, green pea powder, green bean powder, spirulina, proteins derived from vegetables, beans, buckwheat, lentils, pulses, single cell proteins, or combinations thereof.

In an embodiment, the protein is a whey protein.

In an embodiment, the composition further includes a prebiotic selected from the group consisting of acacia gum, alpha glucan, arabinogalactans, beta glucan, dextrans, fructooligosaccharides, fucosyllactose, galactooligosaccharides, galactomannans, gentiooligosaccharides, glucooligosaccharides, guar gum, inulin, isomaltooligosaccharides, lactoneotetraose, lactosucrose, lactulose, levan, maltodextrins, milk oligosaccharides, partially hydrolyzed guar gum, pecticoligosaccharides, resistant starches, retrograded starch, sialooligosaccharides, sialyllactose, soyoligosaccharides, sugar alcohols, xylooligosaccharides, their hydrolysates, or combinations thereof.

In an embodiment, the composition further includes a probiotic selected from the group consisting of probiotics include Aerococcus, Aspergillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, or combinations thereof.

In an embodiment, the composition further includes a phytonutrient selected from the group consisting of flavanoids, allied phenolic compounds, polyphenolic compounds, terpenoids, alkaloids, sulphur-containing compounds, or combinations thereof.

In an embodiment, the phytonutrient is selected from the group consisting of carotenoids, plant sterols, quercetin, curcumin, limonin, or combinations thereof.

In an embodiment, the composition further includes a nucleotide selected from the group consisting of a subunit of deoxyribonucleic acid, a subunit of ribonucleic acid, polymeric forms of DNA and RNA, or combinations thereof. In an embodiment, the nucleotide is an exogenous nucleotide.

In an embodiment, the composition further includes an antioxidant selected from the group consisting of astaxanthin, carotenoids, coenzyme Q10 (“CoQ10”), flavonoids, glutathione, Goji (wolfberry), hesperidin, lactowolfberry, lignan, lutein, lycopene, polyphenols, selenium, vitamin A, vitamin C, vitamin E, zeaxanthin, or combinations thereof.

In an embodiment, the composition further includes a vitamin, wherein the vitamin is selected from the group consisting of vitamin A, Vitamin B1 (thiamine), Vitamin B2 (riboflavin), Vitamin B3 (niacin or niacinamide), Vitamin B5 (pantothenic acid), Vitamin B6 (pyridoxine, pyridoxal, or pyridoxamine, or pyridoxine hydrochloride), Vitamin B7 (biotin), Vitamin B9 (folic acid), and Vitamin B12 (various cobalamins; commonly cyanocobalamin in vitamin supplements), vitamin C, vitamin D, vitamin E, vitamin K, K1 and K2 (i.e., MK-4, MK-7), folic acid, biotin, or combinations thereof.

In an embodiment, the composition further includes a mineral, wherein the mineral is selected from the group consisting of boron, calcium, chromium, copper, iodine, iron, magnesium, manganese, molybdenum, nickel, phosphorus, potassium, selenium, silicon, tin, vanadium, zinc, or combinations thereof.

In still yet another embodiment, nutritional kits including a plurality of compositions having from about 15 to about 35 g protein and guidelines recommending that an athlete consume the composition immediately following concurrent training.

In an embodiment, the plurality of the compositions and the guidelines are together in a package.

In an embodiment, the composition includes from about 20 g to about 30 g protein, or about 25 g protein.

In an embodiment, the composition includes essential amino acids selected from the group consisting of phenylalanine, valine, threonine, tryptophan, isoleucine, methionine, leucine, lysine, histidine, or combinations thereof.

In an embodiment, the composition is enriched with L-[ring-13C6] phenylalanine in an amount up to about 10% by weight of the composition, or up to about 5% by weight of the composition.

In an embodiment, the protein synthesis enhanced is mitochondrial protein synthesis.

In an embodiment, the protein synthesis enhanced is myofibrillar protein synthesis.

The composition may be in a form selected from the group consisting of a solid, a gel, a liquid, a ready-to-mix powder, or combinations thereof. In an embodiment, the composition is a liquid.

In an embodiment, a serving size of the composition is about 500 mL.

In an embodiment, the composition is administered from about 0 to about 30 minutes after concurrent training, or from about 2 to about 15 minutes after concurrent training, or from about 5 to about 10 minutes after concurrent training, or within about 5 minutes after concurrent training.

In an embodiment, the protein is selected from the group consisting of dairy based proteins, plant based proteins, animal based proteins, artificial proteins, or combinations thereof. The dairy based proteins may be selected from the group consisting of casein, caseinates, casein hydrolysate, whey, whey hydrolysates, whey concentrates, whey isolates, milk protein concentrate, milk protein isolate, or combinations thereof. The plant based proteins may be selected from the group consisting of soy protein, pea protein, canola protein, wheat and fractionated wheat proteins, corn proteins, zein proteins, rice proteins, oat proteins, potato proteins, peanut proteins, green pea powder, green bean powder, spirulina, proteins derived from vegetables, beans, buckwheat, lentils, pulses, single cell proteins, or combinations thereof.

In an embodiment, the protein is a whey protein.

In an embodiment, the plurality of compositions further include a prebiotic selected from the group consisting of acacia gum, alpha glucan, arabinogalactans, beta glucan, dextrans, fructooligosaccharides, fucosyllactose, galactooligosaccharides, galactomannans, gentiooligosaccharides, glucooligosaccharides, guar gum, inulin, isomaltooligosaccharides, lactoneotetraose, lactosucrose, lactulose, levan, maltodextrins, milk oligosaccharides, partially hydrolyzed guar gum, pecticoligosaccharides, resistant starches, retrograded starch, sialooligosaccharides, sialyllactose, soyoligosaccharides, sugar alcohols, xylooligosaccharides, their hydrolysates, or combinations thereof.

In an embodiment, the plurality of compositions further include a probiotic selected from the group consisting of probiotics include Aerococcus, Aspergillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, or combinations thereof.

In an embodiment, the plurality of compositions further include a phytonutrient selected from the group consisting of flavanoids, allied phenolic compounds, polyphenolic compounds, terpenoids, alkaloids, sulphur-containing compounds, or combinations thereof.

In an embodiment, the phytonutrient is selected from the group consisting of carotenoids, plant sterols, quercetin, curcumin, limonin, or combinations thereof.

In an embodiment, the plurality of compositions further include a nucleotide selected from the group consisting of a subunit of deoxyribonucleic acid, a subunit of ribonucleic acid, polymeric forms of DNA and RNA, or combinations thereof. In an embodiment, the nucleotide is an exogenous nucleotide.

In an embodiment, the plurality of compositions further include an antioxidant selected from the group consisting of astaxanthin, carotenoids, coenzyme Q10 (“CoQ10”), flavonoids, glutathione, Goji (wolfberry), hesperidin, lactowolfberry, lignan, lutein, lycopene, polyphenols, selenium, vitamin A, vitamin C, vitamin E, zeaxanthin, or combinations thereof.

In an embodiment, the plurality of compositions further include a vitamin, wherein the vitamin is selected from the group consisting of vitamin A, Vitamin B1 (thiamine), Vitamin B2 (riboflavin), Vitamin B3 (niacin or niacinamide), Vitamin B5 (pantothenic acid), Vitamin B6 (pyridoxine, pyridoxal, or pyridoxamine, or pyridoxine hydrochloride), Vitamin B7 (biotin), Vitamin B9 (folic acid), and Vitamin B12 (various cobalamins; commonly cyanocobalamin in vitamin supplements), vitamin C, vitamin D, vitamin E, vitamin K, K1 and K2 (i.e., MK-4, MK-7), folic acid, biotin, or combinations thereof.

In an embodiment, the plurality of compositions further include a mineral, wherein the mineral is selected from the group consisting of boron, calcium, chromium, copper, iodine, iron, magnesium, manganese, molybdenum, nickel, phosphorus, potassium, selenium, silicon, tin, vanadium, zinc, or combinations thereof.

An advantage of the present disclosure is to provide improved methods for enhancing muscle protein synthesis following concurrent training.

Yet another advantage of the present disclosure is to provide programs for enhancing muscle adaptation resulting from concurrent training.

Still yet another advantage of the present disclosure is to provide kits including a plurality of compositions designed to enhance muscle protein synthesis following concurrent training.

Another advantage of the present disclosure is to provide methods for enhancing mitochondrial protein synthesis via administration of protein following concurrent training.

Another advantage of the present disclosure is to provide methods for enhancing myofibrillar protein synthesis via administration of protein following concurrent training.

Additional features and advantages are described herein, and will be apparent from the following Detailed Description and the figures.

### BRIEF DESCRIPTION OF THE FIGURES

FIG. 1 is a schematic representation of the Example of the present disclosure. Subjects reported to the laboratory following an overnight fast and an after initial resting blood sample began a constant infusion of L-[ring-13C6] phenylalanine. 180 minutes after commencement of tracer infusion, a baseline muscle biopsy (vastus lateralis) was obtained, and subjects then completed a concurrent exercise session consisting of resistance exercise (8 sets of 5 leg extension at 80% 1-RM) and endurance exercise (30 minutes cycling at 70% VO₂ peak) separated by 15 minutes. Immediately after the exercise, subjects consumed a 500 mL bolus of protein (25 g whey) or placebo. Additional muscle biopsies were taken at 1 and 4 hours post-exercise.

FIG. 2 illustrates graphs of the (A) plasma insulin, (B) total plasma amino acid, and (C) plasma branched chain amino acid concentrations, for the trial participants when at rest and during 240 minutes of recovery following a concurrent exercise of session resistance exercise (8 sets of 5 leg extension at 80% 1-RM) and endurance exercise (30 minutes cycling at 70% VO₂ peak) and ingestion of either 500 mL placebo or protein beverage immediately post-exercise. Values are mean values±standard deviation. Significantly different (P<0.05) versus (a) rest.

FIG. 3 illustrates graphs of the (A) AktSer473, (B) mammalian target of rapamycin (mTOR) Ser2448, (C) p70S6KThr389, and (D) eukaryotic elongation factor 2 (eEF2) Thr56 phosphorylation, in skeletal muscle of the trial participants when at rest and during 4 hours post-exercise recovery following a concurrent exercise session of resistance exercise (8 sets of 5 leg extension at 80% 1-RM) and endurance exercise (30 minutes cycling at 70% VO₂ peak) and ingestion of either 500 mL placebo or protein beverage immediately post-exercise. Values are expressed relative to α-tubulin and presented in arbitrary units (mean value±standard deviation, n=8). Significantly different (P<0.05) versus (a) rest, (b) 1 hour, and (asterisk) between treatments (placebo vs. protein) at equivalent time-point.

FIG. 4 illustrates graphs of the (A) 5′ adenosine monophosphate-activated protein kinase (AMPK) Thr172, and (B) Glycogen Synthase (GS) Ser641 phosphorylation, in skeletal muscle of the trial participants when at rest and during 4 hours post-exercise recovery following a concurrent exercise session of resistance exercise (8 sets of 5 leg extension at 80% 1-RM) and endurance exercise (30 minutes cycling at 70% VO₂ peak) and ingestion of either 500 mL placebo or protein beverage immediately post-exercise. Values are expressed relative to α-tubulin and presented in arbitrary units (mean value±standard deviation, n=8). Significantly different (P<0.05) versus (a) rest, (b) 1 hour and (asterisk) between treatments (placebo vs. protein) at equivalent time-point.

FIG. 5 illustrates graphs of the (A) Muscle ring finger 1 (“MuRF1”), (B) atrogin, and (C) myostatin messenger ribonucleic acid (“mRNA”) abundance, of the trial participants when at rest and during 4 hours post-exercise recovery following a concurrent exercise session of resistance exercise (8 sets of 5 leg extension at 80% 1-RM) and endurance exercise (30 minutes cycling at 70% VO₂ peak) and ingestion of either 500 mL placebo or protein beverage immediately post-exercise. Values are expressed relative to glyceraldehyde 3-phosphate dehydrogenase (“GAPDH”) and presented in arbitrary units (mean value±standard deviation, n=8). Significantly different (P<0.05) versus (a) rest, (b) 1 hour and (asterisk) between treatments (placebo vs. protein) at equivalent time-point.

FIG. 6 illustrates graphs of the (A) peroxisome proliferator-activated receptor gamma coactivator 1-alpha (“PGC-1α”), (B) hexokinase, and (C) vascular endothelial growth factor mRNA abundance, of the trial participants when at rest and during 4 hour post-exercise recovery following a concurrent exercise session of resistance exercise (8 sets of 5 leg extension at 80% 1-RM) and endurance exercise (30 minutes cycling at 70% VO₂ peak) and ingestion of either 500 mL placebo or protein beverage immediately post-exercise. Values are expressed relative to glyceraldehyde 3-phosphate dehydrogenase (“GAPDH”) and presented in arbitrary units (mean value±standard deviation, n=8). Significantly different (P<0.05) versus (a) rest, (b) 1 hour and (asterisk) between treatments (placebo vs. protein) at equivalent time-point.

FIG. 7 illustrates graphs of the (A) myofibrillar (n=8), and (B) mitochondrial (n=6) protein fractional synthetic rates of the trial participants between 1-4 hour recovery following a concurrent exercise session of resistance exercise (8 sets of 5 leg extension at 80% 1-RM) and endurance exercise (30 minutes cycling at 70% VO₂ peak) and ingestion of either 500 mL placebo or protein (25 g whey protein) beverage immediately post-exercise. Values are expressed as %/hour and presented as individual data with group mean. Significantly different (P<0.05) versus (a) rest, and (asterisk) placebo vs. protein.

### DETAILED DESCRIPTION

As used in this disclosure and the appended claims, the singular forms “a,” “an” and “the” include plural referents unless the context clearly dictates otherwise. Thus, for example, reference to “an amino acid” includes a mixture of two or more amino acids, and the like.

As used herein, “about” is understood to refer to numbers in a range of numerals. Moreover, all numerical ranges herein should be understood to include all integer, whole or fractions, within the range.

As used herein the term “amino acid” is understood to include one or more amino acids. The amino acid can be, for example, alanine, arginine, asparagine, aspartate, citrulline, cysteine, glutamate, glutamine, glycine, histidine, hydroxyproline, hydroxyserine, hydroxytyrosine, hydroxylysine, isoleucine, leucine, lysine, methionine, phenylalanine, proline, serine, taurine, threonine, tryptophan, tyrosine, valine, or combinations thereof.

As used herein, “animal” includes, but is not limited to, mammals, which include but is not limited to, rodents, aquatic mammals, domestic animals such as dogs and cats, farm animals such as sheep, pigs, cows and horses, and humans. Wherein the terms “animal” or “mammal” or their plurals are used, it is contemplated that it also applies to any animals that are capable of the effect exhibited or intended to be exhibited by the context of the passage.

As used herein, the term “antioxidant” is understood to include any one or more of various substances such as beta-carotene (a vitamin A precursor), vitamin C, vitamin E, and selenium that inhibit oxidation or reactions promoted by Reactive Oxygen Species (“ROS”) and other radical and non-radical species. Additionally, antioxidants are molecules capable of slowing or preventing the oxidation of other molecules. Non-limiting examples of antioxidants include carotenoids, coenzyme Q10 (“CoQ10”), flavonoids, glutathione, Goji (wolfberry), hesperidin, lactowolfberry, lignan, lutein, lycopene, polyphenols, selenium, vitamin A, vitamin B₁, vitamin B₆, vitamin B₁₂, vitamin C, vitamin D, vitamin E, zeaxanthin, or combinations thereof.

As used herein, “carbohydrate(s)” are meant to include:

Monosaccharides, which include, but are not limited to, Trioses (such as Ketotriose (Dihydroxyacetone); Aldotriose (Glyceraldehyde)); Tetroses, which include Ketotetrose (such as: Erythrulose) and Aldotetroses (such as Erythrose, Threose); Pentoses, which include Ketopentose (such as Ribulose, Xylulose), Aldopentose (such as Ribose, Arabinose, Xylose, Lyxose), Deoxy sugar (such as Deoxyribose); Hexoses, which include Ketohexose (such as Psicose, Fructose, Sorbose, Tagatose), Aldohexose (such as Allose, Altrose, Glucose, Mannose, Gulose, Idose, Galactose, Talose), Deoxy sugar (such as Fucose, Fuculose, Rhamnose); Heptose (such as Sedoheptulose); Octose; Nonose (such as Neuraminic acid);

Disaccharides, which include, but are not limited to, Sucrose; Lactose; Maltose; Trehalose; Turanose; Cellobiose; kojiboise; nigerose; isomaltose; and palatinose;

Trisaccharides, which include, but are not limited to Melezitose; and Maltotriose;

Oligosaccharides, which include, but are not limited to, corn syrups and maltodextrin; and

Polysaccharides, which include, but are not limited to, glucan (such as dextrin, dextran, beta-glucan), glycogen, mannan, galactan, and starch (such as those from corn, wheat, tapioca, rice, and potato, including Amylose and Amylopectin. The starches can be natural or modified or gelatinized);

or combinations thereof.

Carbohydrates are also understood to include sources of sweeteners such as honey, maple syrup, glucose (dextrose), corn syrup, corn syrup solids, high fructose corn syrups, crystalline fructose, juice concentrates, and crystalline juice.

As used herein, “concurrent training” refers to combined resistance and endurance exercise.

As used herein, “effective amount” is an amount that prevents a deficiency, treats a disease or medical condition in an individual or, more generally, reduces symptoms, manages progression of the diseases or provides a nutritional, physiological, or medical benefit to the individual. A treatment can be patient- or doctor-related.

As used herein, non-limiting examples of sources of ω-3 fatty acids such as α-linolenic acid (“ALA”), docosahexaenoic acid (“DHA”) and eicosapentaenoic acid (“EPA”) include fish oil, krill, poultry, eggs, or other plant or nut sources such as flax seed, walnuts, almonds, algae, modified plants, etc.

As used herein, “food grade micro-organisms” means micro-organisms that are used and generally regarded as safe for use in food.

As used herein, “immediately following” means that an action (e.g., consumption of a protein beverage) takes places from about 0 to about 30 minutes, or from about 2 to about 15 minutes, or from about 5 to 10 minutes, after the activity. In an embodiment, the action is performed within about 5 minutes after the activity.

While the terms “individual” and “patient” are often used herein to refer to a human, the invention is not so limited. Accordingly, the terms “individual” and “patient” refer to any animal, mammal or human having or at risk for a medical condition that can benefit from the treatment.

As used herein, “mammal” includes, but is not limited to, rodents, aquatic mammals, domestic animals such as dogs and cats, farm animals such as sheep, pigs, cows and horses, and humans. Wherein the term “mammal” is used, it is contemplated that it also applies to other animals that are capable of the effect exhibited or intended to be exhibited by the mammal.

The term “microorganism” is meant to include the bacterium, yeast and/or fungi, a cell growth medium with the microorganism, or a cell growth medium in which microorganism was cultivated.

As used herein, the term “minerals” is understood to include boron, calcium, chromium, copper, iodine, iron, magnesium, manganese, molybdenum, nickel, phosphorus, potassium, selenium, silicon, tin, vanadium, zinc, or combinations thereof.

As used herein, a “non-replicating” microorganism means that no viable cells and/or colony forming units can be detected by classical plating methods. Such classical plating methods are summarized in the microbiology book: James Monroe Jay, et al. 2005. Modern Food Microbiology, 7th ed. Springer Science, New York, N.Y., pp. 790. Typically, the absence of viable cells can be shown as follows: no visible colony on agar plates or no increasing turbidity in liquid growth medium after inoculation with different concentrations of bacterial preparations (‘non replicating’ samples) and incubation under appropriate conditions (aerobic and/or anaerobic atmosphere for at least 24 hours). For example, bifidobacteria such as Bifidobacterium longum, Bifidobacterium lactis and Bifidobacterium breve or lactobacilli, such as Lactobacillus paracasei or Lactobacillus rhamnosus, may be rendered non-replicating by heat treatment, in particular low temperature/long time heat treatment.

As used herein, a “nucleotide” is understood to be a subunit of deoxyribonucleic acid (“DNA”) or ribonucleic acid (“RNA”). It is an organic compound made up of a nitrogenous base, a phosphate molecule, and a sugar molecule (deoxyribose in DNA and ribose in RNA). Individual nucleotide monomers (single units) are linked together to form polymers, or long chains. Exogenous nucleotides are specifically provided by dietary supplementation. The exogenous nucleotide can be in a monomeric form such as, for example, 5′-Adenosine Monophosphate (“5′-AMP”), 5′-Guanosine Monophosphate (“5′-GMP”), 5′-Cytosine Monophosphate (“5′-CMP”), 5′-Uracil Monophosphate (“5′-UMP”), 5′-Inosine Monophosphate (“5′-IMP”), 5′-Thymine Monophosphate (“5′-TMP”), or combinations thereof. The exogenous nucleotide can also be in a polymeric form such as, for example, an intact RNA. There can be multiple sources of the polymeric form such as, for example, yeast RNA.

“Nutritional compositions,” or “nutritional products,” as used herein, are understood to include any number of optional additional ingredients, including conventional food additives, for example one or more, acidulants, additional thickeners, buffers or agents for pH adjustment, chelating agents, colorants, emulsifies, excipient, flavor agent, mineral, osmotic agents, a pharmaceutically acceptable carrier, preservatives, stabilizers, sugar, sweeteners, texturizers, and/or vitamins. The optional ingredients can be added in any suitable amount.

As used herein, “phytochemicals” or “phytonutrients” are non-nutritive compounds that are found in many foods. Phytochemicals are functional foods that have health benefits beyond basic nutrition, and are health promoting compounds that come from plant sources. “Phytochemicals” and “Phytonutrients” refers to any chemical produced by a plant that imparts one or more health benefit on the user. Non-limiting examples of phytochemicals and phytonutrients include those that are:

i) phenolic compounds which include monophenols (such as, for example, apiole, carnosol, carvacrol, dillapiole, rosemarinol); flavonoids (polyphenols) including flavonols (such as, for example, quercetin, fingerol, kaempferol, myricetin, rutin, isorhamnetin), flavanones (such as, for example, fesperidin, naringenin, silybin, eriodictyol), flavones (such as, for example, apigenin, tangeritin, luteolin), flavan-3-ols (such as, for example, catechins, (+)-catechin, (+)-gallocatechin, (−)-epicatechin, (−)-epigallocatechin, (−)-epigallocatechin gallate (EGCG), (−)-epicatechin 3-gallate, theaflavin, theaflavin-3-gallate, theaflavin-3′-gallate, theaflavin-3,3′-digallate, thearubigins), anthocyanins (flavonals) and anthocyanidins (such as, for example, pelargonidin, peonidin, cyanidin, delphinidin, malvidin, petunidin), isoflavones (phytoestrogens) (such as, for example, daidzein (formononetin), genistein (biochanin A), glycitein), dihydroflavonols, chalcones, coumestans (phytoestrogens), and Coumestrol; Phenolic acids (such as: Ellagic acid, Gallic acid, Tannic acid, Vanillin, curcumin); hydroxycinnamic acids (such as, for example, caffeic acid, chlorogenic acid, cinnamic acid, ferulic acid, coumarin); lignans (phytoestrogens), silymarin, secoisolariciresinol, pinoresinol and lariciresinol); tyrosol esters (such as, for example, tyrosol, hydroxytyrosol, oleocanthal, oleuropein); stilbenoids (such as, for example, resveratrol, pterostilbene, piceatannol) and punicalagins;

ii) terpenes (isoprenoids) which include carotenoids (tetraterpenoids) including carotenes (such as, for example, α-carotene, β-carotene, γ-carotene, δ-carotene, lycopene, neurosporene, phytofluene, phytoene), and xanthophylls (such as, for example, canthaxanthin, cryptoxanthin, aeaxanthin, astaxanthin, lutein, rubixanthin); monoterpenes (such as, for example, limonene, perillyl alcohol); saponins; lipids including: phytosterols (such as, for example, campesterol, beta sitosterol, gamma sitosterol, stigmasterol), tocopherols (vitamin E), and ω-3, -6, and -9 fatty acids (such as, for example, gamma-linolenic acid); triterpenoid (such as, for example, oleanolic acid, ursolic acid, betulinic acid, moronic acid);

iii) betalains which include Betacyanins (such as: betanin, isobetanin, probetanin, neobetanin); and betaxanthins (non glycosidic versions) (such as, for example, indicaxanthin, and vulgaxanthin);

iv) organo sulfides, which include, for example, dithiolthiones (isothiocyanates) (such as, for example, sulphoraphane); and thiosulphonates (allium compounds) (such as, for example, allyl methyl trisulfide, and diallyl sulfide), indoles, glucosinolates, which include, for example, indole-3-carbinol; sulforaphane; 3,3′-diindolylmethane; sinigrin; allicin; alliin; allyl isothiocyanate; piperine; syn-propanethial-S-oxide;

v) protein inhibitors, which include, for example, protease inhibitors;

vi) other organic acids which include oxalic acid, phytic acid (inositol hexaphosphate); tartaric acid; and anacardic acid; or

vii) combinations thereof.

As used herein, a “prebiotic” is a food substance that selectively promotes the growth of beneficial bacteria or inhibits the growth or mucosal adhesion of pathogenic bacteria in the intestines. They are not inactivated in the stomach and/or upper intestine or absorbed in the gastrointestinal tract of the person ingesting them, but they are fermented by the gastrointestinal microflora and/or by probiotics. Prebiotics are, for example, defined by Glenn R. Gibson and Marcel B. Roberfroid. 1995. Dietary Modulation of the Human Colonic Microbiota Introducing the Concept of Prebiotics. J. Nutr. 125:1401-1412. Non-limiting examples of prebiotics include acacia gum, alpha glucan, arabinogalactans, beta glucan, dextrans, fructooligosaccharides, fucosyllactose, galactooligosaccharides, galactomannans, gentiooligosaccharides, glucooligosaccharides, guar gum, inulin, isomaltooligosaccharides, lactoneotetraose, lactosucrose, lactulose, levan, maltodextrins, milk oligosaccharides, partially hydrolyzed guar gum, pecticoligosaccharides, resistant starches, retrograded starch, sialooligosaccharides, sialyllactose, soyoligosaccharides, sugar alcohols, xylooligosaccharides, or their hydrolysates, or combinations thereof.

As used herein, probiotic micro-organisms (hereinafter “probiotics”) are food-grade microorganisms (alive, including semi-viable or weakened, and/or non-replicating), metabolites, microbial cell preparations or components of microbial cells that could confer health benefits on the host when administered in adequate amounts, more specifically, that beneficially affect a host by improving its intestinal microbial balance, leading to effects on the health or well-being of the host. Salminen S, et al. 1999. Probiotics: how should they be defined? Trends Food Sci. Technol. 10: 107-10. In general, it is believed that these micro-organisms inhibit or influence the growth and/or metabolism of pathogenic bacteria in the intestinal tract. The probiotics may also activate the immune function of the host. For this reason, there have been many different approaches to include probiotics into food products. Non-limiting examples of probiotics include Aerococcus, Aspergillus, Bacillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, or combinations thereof.

The terms “protein,” “peptide,” “oligopeptides” or “polypeptide,” as used herein, are understood to refer to any composition that includes, a single amino acids (monomers), two or more amino acids joined together by a peptide bond (dipeptide, tripeptide, or polypeptide), collagen, precursor, homolog, analog, mimetic, salt, prodrug, metabolite, or fragment thereof or combinations thereof. For the sake of clarity, the use of any of the above terms is interchangeable unless otherwise specified. It will be appreciated that polypeptides (or peptides or proteins or oligopeptides) often contain amino acids other than the 20 amino acids commonly referred to as the 20 naturally occurring amino acids, and that many amino acids, including the terminal amino acids, may be modified in a given polypeptide, either by natural processes such as glycosylation and other post-translational modifications, or by chemical modification techniques which are well known in the art. Among the known modifications which may be present in polypeptides of the present invention include, but are not limited to, acetylation, acylation, ADP-ribosylation, amidation, covalent attachment of a flavanoid or a heme moiety, covalent attachment of a polynucleotide or polynucleotide derivative, covalent attachment of a lipid or lipid derivative, covalent attachment of phosphatidylinositol, cross-linking, cyclization, disulfide bond formation, demethylation, formation of covalent cross-links, formation of cystine, formation of pyroglutamate, formylation, gamma-carboxylation, glycation, glycosylation, glycosylphosphatidyl inositol (“GPI”) membrane anchor formation, hydroxylation, iodination, methylation, myristoylation, oxidation, proteolytic processing, phosphorylation, prenylation, racemization, selenoylation, sulfation, transfer-RNA mediated addition of amino acids to polypeptides such as arginylation, and ubiquitination. The term “protein” also includes “artificial proteins” which refers to linear or non-linear polypeptides, consisting of alternating repeats of a peptide.

Non-limiting examples of proteins include dairy based proteins, plant based proteins, animal based proteins and artificial proteins. Dairy based proteins include, for example, casein, caseinates (e.g., all forms including sodium, calcium, potassium caseinates), casein hydrolysates, whey (e.g., all forms including concentrate, isolate, demineralized), whey hydrolysates, milk protein concentrate, and milk protein isolate. Plant based proteins include, for example, soy protein (e.g., all forms including concentrate and isolate), pea protein (e.g., all forms including concentrate and isolate), canola protein (e.g., all forms including concentrate and isolate), other plant proteins that commercially are wheat and fractionated wheat proteins, corn and it fractions including zein, rice, oat, potato, peanut, green pea powder, green bean powder, and any proteins derived from beans, lentils, and pulses. Animal based proteins may be selected from the group consisting of beef, poultry, fish, lamb, seafood, or combinations thereof.

As used herein, a “synbiotic” is a supplement that contains both a prebiotic and a probiotic that work together to improve the microflora of the intestine.

As used herein, the terms “treatment,” “treat” and “to alleviate” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The term does not necessarily imply that a subject is treated until total recovery. The terms “treatment” and “treat” also refer to the maintenance and/or promotion of health in an individual not suffering from a disease but who may be susceptible to the development of an unhealthy condition, such as nitrogen imbalance or muscle loss. The terms “treatment,” “treat” and “to alleviate” are also intended to include the potentiation or otherwise enhancement of one or more primary prophylactic or therapeutic measure. The terms “treatment,” “treat” and “to alleviate” are further intended to include the dietary management of a disease or condition or the dietary management for prophylaxis or prevention a disease or condition.

As used herein the term “vitamin” is understood to include any of various fat-soluble or water-soluble organic substances (non-limiting examples include vitamin A, Vitamin B1 (thiamine), Vitamin B2 (riboflavin), Vitamin B3 (niacin or niacinamide), Vitamin B5 (pantothenic acid), Vitamin B6 (pyridoxine, pyridoxal, or pyridoxamine, or pyridoxine hydrochloride), Vitamin B7 (biotin), Vitamin B9 (folic acid), and Vitamin B12 (various cobalamins; commonly cyanocobalamin in vitamin supplements), vitamin C, vitamin D, vitamin E, vitamin K, folic acid and biotin) essential in minute amounts for normal growth and activity of the body and obtained naturally from plant and animal foods or synthetically made, pro-vitamins, derivatives, analogs.

In an embodiment, a source of vitamins or minerals can include at least two sources or forms of a particular nutrient. This represents a mixture of vitamin and mineral sources as found in a mixed diet. Also, a mixture may also be protective in case an individual has difficulty absorbing a specific form, a mixture may increase uptake through use of different transporters (e.g., zinc, selenium), or may offer a specific health benefit. As an example, there are several forms of vitamin E, with the most commonly consumed and researched being tocopherols (alpha, beta, gamma, delta) and, less commonly, tocotrienols (alpha, beta, gamma, delta), which all vary in biological activity. There is a structural difference such that the tocotrienols can more freely move around the cell membrane; several studies report various health benefits related to cholesterol levels, immune health, and reduced risk of cancer development. A mixture of tocopherols and tocotrienols would cover the range of biological activity.

The present disclosure relates to methods for enhancing muscle protein synthesis following concurrent training. Specifically, the present disclosure provides methods for enhancing mitochondrial protein synthesis via administration of protein or essential amino acids following concurrent training. More specifically, the present disclosure provides methods for enhancing myofibrillar protein synthesis via administration of protein or essential amino acids following concurrent training.

Physical exercise alters the activity of proteins involved in ‘turning on’ protein synthesis (i.e., signaling molecules), which helps guide which muscle proteins are to be made and when. Similar to the molecular responses, the changes in protein synthesis after a single bout of exercise are largely specific to the exercise task, such as an increased synthesis of proteins involved in strength (i.e., myofibrillar) with resistance exercise and an increase of proteins involved with energy supply (i.e., mitochondrial) with aerobic exercise. Coffey V G, and Hawley J A. 2007. The Molecular Bases of Training Adaptation. Sports Medicine. 37: 737-763.

For physical exercise overtime, these changes in signaling molecule activity and muscle protein synthesis summate over a period of weeks to months (i.e., with training) into physiological adaptations that make an athlete better at a specific exercise task/event.

There is very little information regarding what and how nutrition can support and optimize these training adaptations. In fact, the 2007 Coffee reference mentioned previously does not discuss the role that nutrition plays in supporting or enhancing the adaptation to training. Furthermore, some exercise tasks may have a resistive component followed by an aerobic component (e.g., stop-and-go commonly seen in team sports) and therefore it is completely unknown what changes occur in the molecular signaling as well as the synthesis of different muscle proteins.

There are three main different types of exercise training regimes which include: 1) resistance exercise 2) anaerobic or repeated sprint type exercise and 3) endurance exercise. Each of these exercise training regimes features a divergent training response.

1) Resistance exercise is when subjects undertake explosive movements of weight, with long periods of rest, and is primarily driven by the phosphocreatine and glycolytic energy systems. This system can produce energy quickly, but fatigues quickly. The primary adaptations include increases in muscle mass (hypertrophy) by increased muscle cross-section area through repeated weight lifting training Hakkinen K. 1989. Neuromuscular and hormonal adaptations during strength and power training J. Sports Med. Phys. Fitness. 29:9-26; and Hakkinen K. et. al. 1987. Relationships between training volume, physical performance capacity, and serum hormone concentrations during prolonged training in elite weight lifters. Int. J. Sports Med. 8 Suppl 1:61-65.

2) Repeated sprint type training is anaerobic in nature, involves high-intensity exercise with limited recovery periods, and involves nearly purely carbohydrate metabolism with a large breakdown in muscle glycogen (glycolytic energy production). During these situations of anaerobic energy production, such as high intensity speed training, or sports involving repeated sprints, the increased load on the muscles is accomplished by an increased firing of Type IIa fibers. Finally, at very high workloads, type IIb glycolytic muscle fibers become activated to maintain the high demand of energy provision via anaerobic energy provision. However, during these situations, the high rate of anaerobic energy production exceeds the rate at which it can be oxidized aerobically within the mitochondria, and this leads to the extreme levels of lactate production found in these types of training situations. Spriet L L, Howlett R A, and Heigenhauser G J. 2000. An enzymatic approach to lactate production in human skeletal muscle during exercise. Med. Sci. Sports Exerc. 32: 756-763. Recent studies have looked at the adaptation to repeated sprint training and found that type IIa fibers increase, along with increases in both mitochondria and also some hypertrophy, and increases in the lactate transporters 2. Gibala M J, et al. 2006. Short-term sprint interval versus traditional endurance training: similar initial adaptations in human skeletal muscle and exercise performance. J. Physiol. 575: 901-911.

3) Endurance training is characterized by individuals doing low-intensity training over prolonged periods (e.g., >15 minutes). The energy system represented for endurance training includes the aerobic system, which primarily uses aerobic metabolism of fats and carbohydrates to produce the required energy within the mitochondria when ample oxygen is present. The primary adaptations include increased muscle glycogen stores and glycogen sparing at sub-maximal workloads via increased fat oxidation, enhanced lactate kinetics and morphological alterations, including greater type I fiber per muscle area, and increased capillary and mitochondrial density. Holloszy J O, and Coyle E F. 1984. Adaptations of skeletal muscle to endurance exercise and their metabolic consequences. J. Appl. Physiol. 56: 831-838; and Holloszy J O, Rennie M J, Hickson R C, Conlee R K, and Hagberg J M. 1977. Physiological consequences of the biochemical adaptations to endurance exercise. Ann. N.Y. Acad. Sci. 301: 440-450.

Exercise and nutrition (specifically protein ingestion) are potent stimulators of muscle protein synthesis (“MPS”) with the combination of the two being synergistic. The stimulation of MPS has been shown to be protein fraction specific and dependent on the specific exercise stimulus. Coffey V G, and Hawley J A. 2007. The Molecular Bases of Training Adaptation. Sports Medicine. 37: 737-763. For example, resistance exercise (such as weight lifting) typically stimulates increases in the synthesis of the mitochondrial or myofibrillar (i.e., force generating) protein fraction whereas as aerobic exercise (such as low-intensity, long duration cycling, running, etc.) preferentially increases the mitochondrial (i.e., energy producing) protein fraction; this divergent response provides the basis for training specific adaptations. However, it is not uncommon for sports athletes to perform both resistance and endurance exercise when training for a specific sports performance. This combination of exercise is commonly referred to as concurrent training and has efficacy as the specific adaptations from each mode are beneficial irrespective of the endurance or resistance focus of the sports performance targeted. Wang L, Mascher H, Psilander N, Blomstrand E, and Sahlin K. 2011. Resistance exercise enhances the molecular signaling of mitochondrial biogenesis induced by endurance exercise in human skeletal muscle. J. of Appl. Phys. 111: 1335-1344; and Wilson J, Marin P, Rhea M, Wilson S, Loenneke J, and Anderson J. 2012. Concurrent training: a meta-analysis examining interference of aerobic and resistance exercises. J. Strength Cond. Res. August: 2293-2307. Additionally, concurrent training forms the main component of physical conditioning for team sports players who require a combination of strength and endurance to meet the demands of intermittent “stop and go” sports like soccer and basketball. The potential impact of protein ingestion on the adaptations from concurrent training has not been previously investigated yet this information is important to provide nutritional solutions and advice to individuals who regularly train and compete with this type of training for the most effective recovery from and adaptation to training.

Contraction-induced adaptations in skeletal muscle are largely determined by the mode, volume and intensity of exercise. Coffey V G, and Hawley J A. 2007. The Molecular Bases of Training Adaptation. Sports Medicine. 37: 737-763. Repeated bouts of endurance exercise generates multiple adaptations in skeletal muscle including, but not limited to, increased capillary (Saltin B, and Gollnick P. 1983. Skeletal muscle adaptability. Significance for metabolism and performance. Bethesda, Md.) and mitochondrial density (Holloszy J O. 1967. Biochemical adaptations in muscle. Effects of exercise on mitochondrial oxygen uptake and respiratory enzyme activity in skeletal muscle. J Biol. Chem. 242: 2278-2282), whereas chronic resistance training generally promotes a phenotype of increased myofibrillar protein accretion and cross sectional area of type II fibers. D'Antona G, Lanfranconi F, Pellegrino M A, Brocca L, Adami R, Rossi R, Moro G, Miotti D, Canepari M, and Bottinelli R. 2006. Skeletal muscle hypertrophy and structure and function of skeletal muscle fibres in male body builders. The Journal of Physiology. 570: 611-627; Phillips S M, Tipton K D, Ferrando A A, and Wolfe R R. 1999. Resistance training reduces the acute exercise-induced increase in muscle protein turnover. American Journal of Physiology—Endocrinology And Metabolism. 276: E118-E124. Exercise-nutrient interactions are also critical in determining skeletal muscle adaptation and may have the capacity to modulate the specificity of training response. Hawley J A, Burke L M, Phillips S M, and Spriet L L. 2011. Nutritional modulation of training-induced skeletal muscle adaptations. Journal of Applied Physiology 110: 834-845. Indeed, manipulating carbohydrate availability and/or muscle glycogen stores alter the endurance exercise adaptation response (Bergström J, Hermansen L, Hultman E, and Saltin B. 1967. Diet, muscle glycogen and physical performance. Acta Physiol. Scand. October-November: 140-150; Ivy J L, Katz A L, Cutler C L, Sherman W M, and Coyle E F. 1988. Muscle glycogen synthesis after exercise: effect of time of carbohydrate ingestion. Journal of Applied Physiology. 64: 1480-1485), while protein/amino acid (leucine) supplementation interacts synergistically with resistance exercise to increase muscle protein synthesis. Phillips S M, Hartman J W, and Wilkinson S B. 2005. Dietary Protein to Support Anabolism with Resistance Exercise in Young Men. Journal of the American College of Nutrition. 24: 134S-139S; Rennie M, Edwards R, Halliday D, Matthews D, Wolman S, and Millward D. 1982. Muscle protein synthesis measured by stable isotope techniques in man: the effects of feeding and fasting. Clin. Sci. (Lond) December: 519-523. However, a limited number of studies have investigated the acute adaptation response to the combined effects of endurance and resistance exercise (i.e., concurrent exercise) and, in particular, the interaction with protein ingestion/supplementation.

The cellular mechanisms regulating the specificity of training adaptation within a concurrent training paradigm is undoubtedly complex given the capacity of single mode endurance and resistance training to generate divergent phenotypes (D'Antona G, Lanfranconi F, Pellegrino M A, Brocca L, Adami R, Rossi R, Moro G, Miotti D, Canepari M, and Bottinelli R. 2006. Skeletal muscle hypertrophy and structure and function of skeletal muscle fibres in male body builders. The J. of Phys. 570: 611-627, 2006; and Wilkinson S B, Phillips S M, Atherton P J, Patel R, Yarasheski K E, Tarnopolsky M A, and Rennie M J. 2008. Differential effects of resistance and endurance exercise in the fed state on signalling molecule phosphorylation and protein synthesis in human muscle. The J. of Phys. 586: 3701-3717) and the potential confounding factors of exercise order and recovery between bouts. Wilson and colleagues have reported that endurance exercise inhibits hypertrophy/strength in a volume and frequency dependent manner within a concurrent training paradigm. Wilson J, Marin P, Rhea M, Wilson S, Loenneke J, and Anderson J. 2012. Concurrent training: a meta-analysis examining interference of aerobic and resistance exercises. J. Strength Cond. Res. August: 2293-2307. Applicant also previously demonstrated various cell signaling responses related to translation initiation and mRNA expression of mitochondrial/metabolic and myogenic adaptation following a concurrent exercise bout in the fasted state. Coffey V G, Jemiolo B, Edge J, Garnham A P, Trappe S W, and Hawley J A. 2009. Effect of consecutive repeated sprint and resistance exercise bouts on acute adaptive responses in human skeletal muscle. Am. J. of Phys.—Regulatory, Integrative and Comparative Physiology. 297: R1441-R1451; Coffey V G, Pilegaard H, Garnham A P, O'Brien B J, and Hawley J A. 2009. Consecutive bouts of diverse contractile activity alter acute responses in human skeletal muscle. J. of Appl. Phys. 106: 1187-1197. Interestingly, comparable increased rates of myofibrillar and mitochondrial synthesis were recently shown following concurrent resistance and endurance exercise when compared to each mode in isolation in sedentary middle-aged men. Donges C E, Burd N A, Duffield R, Smith G C, West D W D, Short M J, Mackenzie R, Plank L D, Shepherd P R, Phillips S M, and Edge J A. 2012. Concurrent resistance and aerobic exercise stimulates both myofibrillar and mitochondrial protein synthesis in sedentary middle-aged men. J. of Appl. Phys. 112: 1992-2001. Therefore, while the molecular profile generated by an acute bout of concurrent training has yet to be clearly established, the possibility exists that successive resistance and endurance exercise may have the capacity to promote both myofibrillar and mitochondrial protein synthesis.

Consumption of high-quality protein in close temporal proximity to resistance exercise enhances translation initiation signaling and maximally stimulates rates of muscle protein synthesis. Koopman R, Pennings B, Zorenc A H G, and van Loon L J C. 2007. Protein Ingestion Further Augments S6K1 Phosphorylation in Skeletal Muscle Following Resistance Type Exercise in Males. The Journal of Nutrition. 137: 1880-1886; and Moore D R, Robinson M J, Fry J L, Tang J E, Glover E I, Wilkinson S B, Prior T, Tarnopolsky M A, and Phillips S M. 2009. Ingested protein dose response of muscle and albumin protein synthesis after resistance exercise in young men. The American Journal of Clinical Nutrition. 89: 161-168. Likewise, protein feeding following endurance exercise can increase the transcriptional profile of mitochondrial-related genes. Rowlands D S, Thomson J S, Timmons B W, Raymond F, Fuerholz A, Mansourian R, Zwahlen M-C, Métairon S, Glover E, Stellingwerff T, Kussmann M, and Tarnopolsky M A. 2011. Transcriptome and translational signaling following endurance exercise in trained skeletal muscle: impact of dietary protein. Physiological Genomics. 43: 1004-1020. To date, no studies have determined the effect of protein ingestion following concurrent exercise on the acute myofibrillar and mitochondrial protein synthesis rates in skeletal muscle. Indeed, beyond pure endurance/aerobic exercise and pure resistance exercise, there exists no evidence that the established beneficial effects of protein ingestion on enhancing muscle protein synthesis following either resistance or endurance exercise occurs when protein is consumed following concurrent exercise (i.e., combined resistance exercise then endurance exercise). Accordingly, the present disclosure examines the acute effects of protein ingestion on rates of myofibrillar and mitochondrial protein synthesis in association with selected cellular/molecular responses following a bout of consecutive resistance exercise and endurance exercise (e.g., cycling).

Specifically, an advantage of the present disclosure is that it provides the same beneficial outcome as consuming high-quality whey protein in close proximity (within 30 minutes) to endurance and resistance exercise in isolation. The present disclosure provides a unique outcome that is highly applicable to sports nutrition consumers that habitually perform a combination of exercise types (resistance and endurance exercise) in a given training session. Additionally, the evidence also provides support that the present methods reduce the negative effects of performing these different exercise types sequentially. Namely, previous evidence has demonstrated a reduction of specific muscle protein synthesis when resistance and endurance exercise are performed consecutively. For example, performing endurance exercise (e.g., cycling, running) immediately following resistance exercise (e.g., weight-lifting) reduces strength specific adaptations to the initial resistance training. The present disclosure demonstrates an attenuation of this particular response with a reduction of cellular signaling associated with muscle breakdown.

In a first aspect, the present disclosure provides methods of enhancing muscle protein synthesis following physical exertion comprising administering to a human a composition comprising from about 15 to about 35 g protein immediately following concurrent training.

It has surprisingly been found that the addition of protein or essential amino acids following concurrent exercise has the capacity of enhancing mitochondrial protein synthesis.

It has surprisingly been found that the addition of protein or essential amino acids following concurrent exercise has the capacity of enhancing myofibrillar protein synthesis. Myofibrillar protein is the specific protein responsible for muscle hypertrophy (growth).

Physical exercise provides a stimulus to the body that triggers a cascade of molecular signals that lead to changes in gene expression and the synthesis of proteins specific to the exercise stimulus. The physical adaptation that results from chronic training is believed to be a direct result of the accumulation of these proteins after multiple bouts of acute exercise.

A clear beneficial effect of the present methods is shown on anabolic signaling and muscle protein synthesis following concurrent exercise and subsequent consumption of protein or essential amino acids. Recommendations are to take protein and carbohydrate in close temporary timing to strenuous training sessions. There are well documented advantages of supplementation of nutrients after resistance and endurance training, but not with concurrent training.

It has also been found that when nutrition can improve the adaptations to a single training session by only a small percentage over a period of weeks or months this could have a major effect on training adaptations and thus performance.

In another embodiment, a program for enhancing muscle adaptation resulting from concurrent training is provided. The program includes providing nutrition and guidance on training to an athlete to improve the muscle protein synthesis. The program further includes providing a composition including from about 15 g to about 35 g protein; and providing guidelines for consumption including a recommendation of the amount of the composition to consume immediately following concurrent training based on a training regimen of the athlete, and providing guidance on training regimen.

In a further aspect, the present disclosure relates to a nutritional kit comprising a plurality of compositions including from about 15 g to about 35 g protein and guidelines recommending that an athlete consume the composition immediately following concurrent training. The present disclosure also relates to a use of a composition including protein or essential amino acids and carbohydrates for improving muscle protein synthesis wherein the use is in connection with concurrent training.

Furthermore, as described above, concurrent training includes an anaerobic component that involves nearly purely carbohydrate metabolism with a large breakdown in muscle glycogen. In an embodiment, nutritional recommendations are for at least 1 to 1.5 g of carbohydrate per kg body mass (a total of 50 to 75 g carbohydrate) to be consumed in the first several hours after this type of exercise training.

The present disclosure also provides ways in which individuals can enhance recovery from and adaptation to exercise to allow athletes to “get more out of their training” The target athletes are training for strength and endurance, and/or team sports.

In the present disclosure, Applicant examines the acute effects of protein ingestion on rates of myofibrillar and mitochondrial protein synthesis in association with selected cellular/molecular responses measured directly by muscle biopsy sampling following a bout of consecutive resistance exercise and endurance exercise (e.g., cycling). This order of concurrent exercise training has been shown to negatively impact muscle protein synthesis, therefore Applicant hypothesized that protein ingestion would enhance anabolic and metabolic signaling and subsequent protein synthesis during the early recovery period following concurrent training preventing these negative effects when endurance exercise is performance immediately following strength/muscle hypertrophic resistance exercise.

To investigate the effects of protein ingestion described above, Applicant performed a randomized cross-over, double-blind study that is described in greater detail in the Examples set forth below. Generally, subjects (n=8) reported to a laboratory after an overnight fast on two separate occasions and consumed a 500 ml beverage of either placebo (water and artificial sweetener) or a protein beverage (25 g whey protein) immediately following exercise. Exercise was comprised of resistance (8×5 leg extensions, 80% 1-RM) following by endurance (30 minutes of cycling at 70% VO₂ peak). A primed constant infusion of ring-[13C6]phenylalanine in conjunction with muscle biopsies was used to measure muscle protein synthesis in the myofibrillar (force-generating) and mitochondrial (energy-producing) protein fractions over 4 hours of post-exercise recovery. Changes in the phosphorylation of intracellular signaling proteins involved in mRNA translation (i.e., ‘turning on’ protein synthesis) were measured by Western blot analysis as a surrogate for their activity levels.

Applicant surprisingly found that protein ingestion resulted in a 67% greater myofibrillar protein synthetic rate during the post-exercise recovery period as compared to the placebo condition. This data was in line with the greater phosphorylation (and presumably activity) of candidate signaling proteins within the important regulatory mTOR growth pathway (e.g., AktSer473, mTORSer2448) suggesting an increased rate of mRNA translation prior to muscle protein synthesis. Post-exercise rates of myofibrillar protein synthesis increased above rest in both trials (75-145%), but were higher with PRO showing an additional benefit to protein ingestion above exercise induced responses. Additionally, protein supplementation attenuated the exercise induced muscle proteolysis/catabolism as measured by phosphorylation of signaling proteins linked to the breakdown of muscle protein (e.g., MuRF1, Atrogin-1).

Applicant also found that mitochondrial protein synthesis did not change from baseline with either exercise or protein supplementation, which suggests that either the exercise performed was not sufficient to induced synthesis of this muscle protein fraction or the combination of resistance and endurance exercise may ameliorate mitochondrial protein synthesis.

This data demonstrates the impact of consuming a source of protein immediately following a period of concurrent exercise training. Previously, it was established that the combination of resistance and endurance exercise in close proximity attenuates adaptations assessed as increases in muscle protein synthesis. The novel findings in the present disclosure provide support for protein supplementation following resistance and endurance exercise to allow anabolic adaptation and promotion/protection of muscle mass with reduced potential inference effects associated with endurance exercise on muscle hypertrophy.

Adaptations to concurrent resistance and endurance exercise may be ‘compromised’ when compared with training for either exercise mode alone (Hickson R. 1980. Interference of strength development by simultaneously training for strength and endurance. Eur. J. Appl. Physiol. Occup. Physiol. 45: 255-263; and Wilson J, Marin P, Rhea M, Wilson S, Loenneke J, and Anderson J. 2007. Concurrent training: a meta-analysis examining interference of aerobic and resistance exercises. J. Strength Cond. Res. August: 2293-2307). The results from the studies of the present disclosure show that, in moderately trained individuals, the combined effects of resistance and endurance exercise result in elevated rates of myofibrillar but not mitochondrial protein synthesis. Applicant has also found, for the first time, that protein ingestion promotes insulin/insulin-like growth factor (“IGF”) pathway signaling and myofibrillar protein synthesis, but does not enhance mitochondrial protein synthesis rates during the early recovery period following consecutive resistance exercise and cycling. In addition, the studies of the present disclosure provide new information to demonstrate that post-exercise protein ingestion attenuates mRNA expression of markers of muscle catabolism following a concurrent training session.

Athletes from a variety of sports undertake resistance and endurance training concurrently to enhance both anabolic/growth and metabolic/oxidative adaptations in skeletal muscle. As such, concurrent training presents a unique integration of divergent contractile activity. The primary novel finding of the present study was that a single bout of concurrent training promoted an adaptation response favoring muscle anabolism in moderately trained males, and post-exercise protein supplementation preferentially enhanced rates of myofibrillar but not mitochondrial protein synthesis. Donges and colleagues have recently shown that a concurrent training bout was capable of up regulating translational signaling, and myofibrillar and mitochondrial protein synthesis in untrained, middle-aged subjects to a similar extent as resistance and endurance exercise bouts performed in isolation. Donges C E, Burd N A, Duffield R, Smith G C, West D W D, Short M J, Mackenzie R, Plank L D, Shepherd P R, Phillips S M, and Edge J A. 2012. Concurrent resistance and aerobic exercise stimulates both myofibrillar and mitochondrial protein synthesis in sedentary middle-aged men. Journal of Applied Physiology. 112: 1992-2001. The results of the present study provide support for this exercise-mediated effect on the myofibrillar fraction of skeletal muscle with placebo ingestion, but failed to elevate rates of mitochondrial protein synthesis in our subjects, as will be shown in the Examples below.

Enhanced rates of myofibrillar protein synthesis following resistance exercise with post-exercise protein ingestion are well established (Burd N A, Tang J E, Moore D R, and Phillips S M. 2009. Exercise training and protein metabolism: influences of contraction, protein intake, and sex-based differences. Journal of Applied Physiology 106: 1692-1701; and Moore D R, Robinson M J, Fry J L, Tang J E, Glover E I, Wilkinson S B, Prior T, Tarnopolsky M A, and Phillips S M. 2009. Ingested protein dose response of muscle and albumin protein synthesis after resistance exercise in young men. The American Journal of Clinical Nutrition 89: 161-168). However, this is the first investigation to report increased rates of myofibrillar synthesis with protein supplementation compared to placebo during the acute post-exercise recovery period following concurrent resistance exercise and cycling. Therefore, the findings suggest that resistance exercise generates a sufficient adaptive signal to retain the capacity to stimulate myofibrillar protein synthesis despite a subsequent bout of endurance exercise. Such an acute response would be expected to ultimately result in muscle hypertrophy with repeated bouts of resistance exercise in a chronic concurrent training program.

It has previously been demonstrated that a similar selective increase in myofibrillar protein synthesis rate in response to protein-carbohydrate co-ingestion following a high-intensity repeated sprint protocol. Coffey V, Moore D, Burd N, Rerecich T, Stellingwerff T, Gamham A, Phillips S, and Hawley J. 2011. Nutrient provision increases signaling and protein synthesis in human skeletal muscle after repeated sprints. European Journal of Applied Physiology. 111: 1473-1483. Given the high load (0.75 Nm/kg) and subsequent mechanical force required to complete maximal sprint cycling repetitions, the overload stimulus in previous study may be considered resistance-like exercise that might promote a modest hypertrophy response with protein ingestion. Id. However, Breen and co-workers have also recently reported increases in rates of myofibrillar, but not mitochondrial, protein fractional synthetic rates when carbohydrate-protein was co-ingested compared to carbohydrate feeding alone following 90 minutes of steady state cycling at ˜75% VO₂max. Breen L, Philp A, Witard O C, Jackman S R, Selby A, Smith K, Baar K, and Tipton K D. 2011.

The influence of carbohydrate-protein co-ingestion following endurance exercise on myofibrillar and mitochondrial protein synthesis. The Journal of Physiology. 589: 4011-4025. While some increase in muscle mass in untrained/sedentary individuals likely occurs with contractile overload per se (Harber M P, Konopka A R, Undem M K, Hinkley J M, Minchev K, Kaminsky L A, Trappe T A, and Trappe S W. 2012. Aerobic exercise training induces skeletal muscle hypertrophy and age-dependent adaptations in myofiber function in young and older men. Journal of Applied Physiology), endurance exercise does not induce substantial hypertrophy (Hickson R. 1980. Interference of strength development by simultaneously training for strength and endurance. Eur. J. Appl. Physiol. Occup. Physiol. 45: 255-263, 1980; and Wilson J, Marin P, Rhea M, Wilson S, Loenneke J, and Anderson J. 2012. Concurrent training: a meta-analysis examining interference of aerobic and resistance exercises. J. Strength Cond. Res. August: 2293-2307) and Breen and colleagues postulate that a potential mechanism for the increase in myofibrillar protein synthesis following prolonged endurance exercise and protein ingestion was repair and remodeling of muscle fibers. In contrast, Donges and co-workers have reported an endurance exercise bout combined with post-exercise protein ingestion failed to increase myofibrillar protein synthesis above rest compared with a resistance exercise and concurrent training bout. Donges C E, Burd N A, Duffield R, Smith G C, West D W D, Short M J, Mackenzie R, Plank L D, Shepherd P R, Phillips S M, and Edge J A. 2012. Concurrent resistance and aerobic exercise stimulates both myofibrillar and mitochondrial protein synthesis in sedentary middle-aged men. Journal of Applied Physiology. 112: 1992-2001. Whether the myofibrillar synthetic response observed in the present study is exclusively the result of the resistance exercise or some interaction with the endurance exercise remains unclear. Regardless, the enhanced protein synthesis with protein ingestion is undoubtedly beneficial for retaining/augmenting muscle mass and promoting adaptation with concurrent training.

The results of the studies described below demonstrate variable rates of mitochondrial protein synthesis that failed to increase following the concurrent training bout with either treatment. Previous studies in untrained or sedentary subjects have shown an increase in mitochondrial protein synthesis regardless of the mode of exercise i.e., resistance, endurance or concurrent exercise bouts. Burd N A, Andrews R J, West D W D, Little J P, Cochran A J R, Hector A J, Cashaback J G A, Gibala M J, Potvin J R, Baker S K, and Phillips S M. 2012. Muscle time under tension during resistance exercise stimulates differential muscle protein sub-fractional synthetic responses in men. The Journal of Physiology. 590: 351-362; Donges C E, Burd N A, Duffield R, Smith G C, West D W D, Short M J, Mackenzie R, Plank L D, Shepherd P R, Phillips S M, and Edge J A. 2012. Concurrent resistance and aerobic exercise stimulates both myofibrillar and mitochondrial protein synthesis in sedentary middle-aged men. Journal of Applied Physiology. 112: 1992-2001; and Wilkinson S B, Phillips S M, Atherton P J, Patel R, Yarasheski K E, Tarnopolsky M A, and Rennie M J. Differential effects of resistance and endurance exercise in the fed state on signalling molecule phosphorylation and protein synthesis in human muscle. 2008. The Journal of Physiology. 586: 3701-3717. Consequently, it is suggested that the training status of subjects in the present study may have required a greater overload stimulus to generate an acute increase in mitochondrial protein synthesis. Indeed, Breen and co-workers determined the effect of protein ingestion on muscle protein synthesis in well-trained cyclists and also failed to observe any effect on mitochondrial FSR. Breen L, Philp A, Witard O C, Jackman S R, Selby A, Smith K, Baar K, and Tipton K D. 2011. The influence of carbohydrate-protein co-ingestion following endurance exercise on myofibrillar and mitochondrial protein synthesis. The Journal of Physiology. 589: 4011-4025. Rowlands and co-workers reported an enhanced mitochondrial transcriptome associated with protein ingestion following endurance exercise, an effect that was only evident late (48 hours) but not early (3 hours) in the post-exercise period. Rowlands D S, Thomson J S, Timmons B W, Raymond F, Fuerholz A, Mansourian R, Zwahlen M-C, Métairon S, Glover E, Stellingwerff T, Kussmann M, and Tarnopolsky M A. 2011. Transcriptome and translational signaling following endurance exercise in trained skeletal muscle: impact of dietary protein. Physiological Genomics 43: 1004-1020. Therefore, it cannot be ruled out that the quantification of mitochondrial protein synthesis later in recovery (e.g., 24 hours) may have revealed differences in the adaptation response to exercise and protein ingestion.

The enhanced myofibrillar protein synthesis was associated with increases in the phosphorylation status of signaling proteins that regulate translation initiation and elongation. It was previously demonstrated that a similar time course for Akt-mTOR-S6K phosphorylation during the early recovery period following single bouts of resistance exercise and cycling. Camera D, Edge J, Short M, Hawley J, and Coffey V. 2010. Early time course of Akt phosphorylation after endurance and resistance exercise. Med. Sci. Sports Exerc. October: 1843-1852. Others have also previously shown endurance and resistance exercise in isolation activate the insulin/IGF signaling pathway. Benziane B, Burton T J, Scanlan B, Galuska D, Canny B J, Chibalin A V, Zierath J R, and Stepto N K. 2008. Divergent cell signaling after short-term intensified endurance training in human skeletal muscle. American Journal of Physiology—Endocrinology And Metabolism. 295: E1427-E1438; and Moore D R, Robinson M J, Fry J L, Tang J E, Glover E I, Wilkinson S B, Prior T, Tarnopolsky M A, and Phillips S M. 2009. Ingested protein dose response of muscle and albumin protein synthesis after resistance exercise in young men. The American Journal of Clinical Nutrition. 89: 161-168. Collectively, these findings indicate specific translational processes in skeletal muscle are not an important factor determining the specificity of training adaptation. More recently, a concurrent training bout has been shown to enhance Akt/mTOR-mediated signaling responses. Lundberg T, Fernandez-Gonzalo R, Gustafsson T, and Tesch P. 2012. Aerobic Exercise Alters Skeletal Muscle Molecular Responses to Resistance Exercise. Med Sci Sports Exerc.; and Wang L, Mascher H, Psilander N, Blomstrand E, and Sahlin K. 2011. Resistance exercise enhances the molecular signaling of mitochondrial biogenesis induced by endurance exercise in human skeletal muscle. Journal of Applied Physiology. 111: 1335-1344. The results of the present study extend these findings by demonstrating that protein ingestion can augment Akt-mTOR-S6K phosphorylation following concurrent training Consequently, Akt-mTOR-S6K signaling may be indicative of nutrient sensitivity and/or muscle overload but fails to discriminate between divergent contraction stimuli. Exercise also generated a decrease in phosphorylation (activation) of the peptide chain elongation factor eEF2 although there were no differences between treatments indicating it may be unresponsive to protein ingestion. Thus, nutrient-mediated increases in muscle protein synthesis following exercise are likely due in part to enhanced translation initiation rather than elongation.

The AMPK has been implicated in repressing anabolic signaling and protein synthesis in skeletal muscle via inhibition of mTOR-mediated signaling to initiate translation. Dreyer H C, Fujita S, Cadenas J G, Chinkes D L, Volpi E, and Rasmussen B B. 2006. Resistance exercise increases AMPK activity and reduces 4E-BP1 phosphorylation and protein synthesis in human skeletal muscle. The Journal of Physiology. 576: 613-624; and Gwinn D M, Shackelford D B, Egan D F, Mihaylova M M, Mery A, Vasquez D S, Turk B E, and Shaw R J. 2008. AMPK Phosphorylation of Raptor Mediates a Metabolic Checkpoint. Molecular Cell. 30: 214-226. However, post-exercise increases in AMPKThr172 phosphorylation in the present studies were modest and were concomitant with increases in mTOR phosphorylation. This may reflect an inability of the concurrent exercise session to significantly disrupt cell energy status to a level required to modulate AMPK signaling despite changes in glycogen metabolism with exercise. Coffey V G, Pilegaard H, Garnham A P, O'Brien B J, and Hawley J A. 2009. Consecutive bouts of diverse contractile activity alter acute responses in human skeletal muscle. Journal of Applied Physiology. 106: 1187-1197. Nonetheless, previous research has previously failed to observe an AMPK-associated inhibition of translation initiation signaling or protein synthesis during recovery from exercise in human studies and such a causal relationship has yet to be clearly established in vivo human muscle.

A novel finding of the present study was the attenuated mRNA responses of genes associated with muscle proteolysis and catabolism. MuRF1 and Atrogin-1 mRNA expression was elevated above rest following the concurrent training bout, however this increase was attenuated with protein ingestion. Harber and colleagues previously showed a similar effect on MuRF1 mRNA abundance with ingestion of a protein/carbohydrate supplement following 60 minutes of cycling and Borgenvik and co-workers demonstrated an amino acid-enriched beverage decreased MuRF1 protein levels at rest and after a resistance exercise bout. Harber M P, Konopka A R, Jemiolo B, Trappe S W, Trappe T A, and Reidy P T. 2010. Muscle protein synthesis and gene expression during recovery from aerobic exercise in the fasted and fed states. American Journal of Physiology—Regulatory, Integrative and Comparative Physiology. 299: R1254-R1262; and Borgenvik M, Apró W, and Blomstrand E. 2012. Intake of branched-chain amino acids influences the levels of MAFbx mRNA and MuRF-1 total protein in resting and exercising human muscle. American Journal of Physiology—Endocrinology And Metabolism. 302: E510-E521. Therefore, coordinated attenuation in MuRF1 and Atrogin-1 expression with provision of exogenous amino acids may have provided substrate for muscle remodeling/hypertrophy that might otherwise be achieved through muscle breakdown following exercise in the fasted state. There were no differences between treatments in myostatin mRNA expression during the acute recovery period. Reduced myostatin expression has been demonstrated following an acute bout of endurance (Lundberg T, Fernandez-Gonzalo R, Gustafsson T, and Tesch P. 2012. Aerobic Exercise Alters Skeletal Muscle Molecular Responses to Resistance Exercise. Med. Sci. Sports Exerc.) and resistance (Camera D, West D, Burd N, Phillips S, Garnham A, Hawley J, and Coffey V. 2012. Low Muscle Glycogen Concentration Does Not Suppress the Anabolic Response to Resistance Exercise. J. Appl. Physiol. May 24. [Epub ahead of print]; and Lundberg T, Fernandez-Gonzalo R, Gustafsson T, and Tesch P. 2012. Aerobic Exercise Alters Skeletal Muscle Molecular Responses to Resistance Exercise. Med. Sci. Sports Exerc.) exercise, and it appears myostatin mRNA expression is responsive to contraction per se rather than a specificity of training response and/or nutrient availability. There were comparable increases in mRNA abundance of metabolic/mitochondrial proteins following the consecutive resistance and endurance exercise bouts but protein ingestion failed to induce any noteworthy increase in PGC-1α, hexokinase or VEGF mRNA levels. Accordingly, while concurrent training is capable of generating an adaptive mRNA profile supportive of mitochondrial, metabolic and angiogenic processes in skeletal muscle, this response is not enhanced by amino acid provision.

In order to maximize muscle protein synthesis and enhance adaptations to concurrent exercise, the present disclosure provides methods that provide athletes with a product that contains from about 20 g to about 35 g, or from about 20 g to about 30 g of protein, or 26 g protein, immediately following concurrent training. In an embodiment, the products are consumed within about 0 to about 30 minutes of the exercise.

In an embodiment, the composition includes carbohydrates and protein or essential amino acids, in a carbohydrate to protein ratio in the range from about 1:1 to about 3:1, or in a ratio of about 2:1.

To maximize post-exercise glycogen resynthesis for carbohydrate intake, a recommended consumption amount of carbohydrates would be from about 1 to about 1.5 g CHO/kg.

In an embodiment, the composition includes a total protein dose from about 10 g to about 50 g protein, or from about 20 g to about 30 g protein, or about 25 g protein. The protein or amino acid may constitute from about 20% to about 40% by weight, or about 30% by weight, of the solids in the final composition.

Moreover, the composition can be made so that there is a consistent and countable quantity of protein per single dose, for example, between about 2 grams to about 4 grams per dose. In an embodiment, the composition includes a protein or essential amino acid content from about 2 grams to about 2.5 grams.

The composition may be in the form of a solid product, a gel, a liquid, or a ready to mix powder. In an embodiment, the composition is a protein beverage.

The protein-based composition can also contain a discrete amount of fat in one or more products to provide any suitable amount of energy to an athlete. For example, each of the compositions can provide a fat amount up to about 9 g/300 cal. In another example, the compositions can provide about 11 g/360 cal. Each of the compositions can also provide a saturated fat amount up to 4 g/300 cal or more. In an embodiment, the percentage of energy (e.g., in the form of calories) coming from fat can be up to about 25%.

In an embodiment, the protein-based composition includes an amount of fat ranging from about 10% to about 40% by weight, or about 30% by weight, of the protein-based product.

The presence of proteins and/or fats in the nutritional compositions of the present disclosure has the advantage in that it is possible to provide an athlete with more complete nutrition during performance. For the protein source, any suitable dietary protein may be used such as, for example, animal proteins (e.g. milk proteins, meat proteins and egg proteins); dietary proteins including, but not limited to dairy protein (such as casein, caseinates (e.g., all forms including sodium, calcium, potassium caseinates), casein hydrolysates, whey (e.g., all forms including concentrate, isolate, demineralized), whey hydrolysates, milk protein concentrate, and milk protein isolate)), vegetable proteins (e.g. soy protein, wheat protein, rice protein, and pea protein); mixtures of free amino acids; or combinations thereof. Milk proteins, such as casein and whey milk proteins, and soy proteins are particularly preferred. In an embodiment, the protein source is selected from the group consisting of whey, chicken, corn, caseinate, wheat, flax, soy, carob, pea, or combinations thereof.

The proteins may be intact or hydrolyzed or a mixture of intact and hydrolyzed proteins. It may be desirable to supply partially hydrolyzed proteins (e.g., degree of hydrolysis between 2 and 20%), for example, for athletes believed to be at risk of developing cows' milk allergy. Generally, at least partially hydrolyzed proteins are easier and faster to metabolize by the body. This is in particular true for amino acids. In an embodiment, the protein-based product contains single/essential amino acids such as, for example, leucine, valine and/or isoleucine.

The protein-based product can also include a protein blend comprising, for example, soy protein isolates, whey protein isolates and calcium caseinate. An example of the protein blend is the Tri-source™ protein blend. In an embodiment, however, the protein in the present compositions is whey protein.

In an embodiment of the present disclosure, the essential amino acids include added leucine. In the present context, leucine is an essential amino acid (“EAA”), found as part of the family of branched chain amino acids (“BCAA”). Ingestion of essential EAA stimulates the synthesis of skeletal muscle proteins with the branched-chain amino acids leucine, isoleucine, and valine suggested to play a critical role in this response. Of the BCAA, leucine has been investigated for its anabolic properties in many different tissues, including muscle. It is well established in cell culture and rat models that leucine increases the formation, and hence activation, of specific proteins that are involved in “turning on” protein synthesis.

Advantageously, a total dose of essential amino acid dose from about 5 g to about 25 g blend of EAA that mimics the EAA in high quality proteins, or 10 g EAA is used in a composition according to the present disclosure. In an embodiment, a composition includes leucine in a total dose of up to about 25 g.

In an embodiment, the protein-based composition is enriched to up to about 10%, or up to about 7%, or up to about 5%, or up to about 3% L-[ring-13C6] phenylalanine. In an embodiment, the protein-based composition is enriched to up to about 5% L-[ring-13C6] phenylalanine.

The fat source has the advantage in providing for an improved mouth feel. Any fat source is suitable. For example, animal or plant fats may be used. To increase the nutritional value, ω3-unsaturated and ω6-unsaturated fatty acids may comprise the fat source. The fat source may also contain long chain fatty acids and/or medium chain fatty acids. For example, milk fat, canola oil, almond butter, peanut butter, corn oil and/or high-oleic acid sunflower oil may be used.

The present nutritional compositions may also include other beneficial or functional ingredients. For example, the nutritional compositions may further include one or more prebiotics. The prebiotics may be selected from the group consisting of acacia gum, alpha glucan, arabinogalactans, beta glucan, dextrans, fructooligosaccharides, galactooligosaccharides, galactomannans, gentiooligosaccharides, glucooligosaccharides, guar gum, inulin, isomaltooligosaccharides, lactosucrose, lactulose, levan, maltodextrins, partially hydrolyzed guar gum, pecticoligosaccharides, retrograded starch, soyoligosaccharides, sugar alcohols, xylooligosaccharides, or combinations thereof.

In an embodiment, the nutritional compositions further include one or more probiotics selected from the group consisting of Aerococcus, Aspergillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, or combinations thereof.

The nutritional compositions may also include a source of fiber, fiber or a blend of different types of fiber. The fiber blend may contain a mixture of soluble and insoluble fibers. Soluble fibers may include, for example, fructooligosaccharides, acacia gum, inulin, etc. Insoluble fibers may include, for example, pea outer fiber.

In an embodiment, any suitable carbohydrate may be used in the present nutritional compositions including, but not limited to, sucrose, lactose, glucose, fructose, corn syrup solids, maltodextrin, modified starch, amylose starch, tapioca starch, corn starch, or combinations thereof.

In another embodiment, the nutritional composition further includes one or more amino acids. Non-limiting examples of amino acids include isoleucine, alanine, leucine, asparagine, lysine, aspartate, methionine, cysteine, phenylalanine, glutamate, threonine, glutamine, tryptophan, glycine, valine, proline, serine, tyrosine, arginine, citrulline, histidine, or combinations thereof.

In an embodiment, the nutritional composition further includes one or more synbiotics, phytonutrients and/or antioxidants. The antioxidants may be selected from the group consisting of carotenoids, coenzyme Q10 (“CoQ10”), flavonoids, glutathione, Goji (Wolfberry), hesperidin, Lactowolfberry, lignan, lutein, lycopene, polyphenols, selenium, vitamin A, vitamin B1, vitamin B6, vitamin B12, vitamin C, vitamin D, vitamin E, or combinations thereof.

In an embodiment, the nutritional composition further includes one or more vitamins and minerals. Non-limiting examples of vitamins include Vitamins A, B-complex (such as B-1, B-2, B-6 and B-12), C, D, E and K, niacin and acid vitamins such as pantothenic acid and folic acid, biotin, or combinations thereof. Non-limiting examples of minerals include calcium, iron, zinc, magnesium, iodine, copper, phosphorus, manganese, potassium, chromium, molybdenum, selenium, nickel, tin, silicon, vanadium, boron, or combinations thereof.

Other optional ingredients can be added to make the nutritional composition sufficiently palatable. For example, the nutritional compositions of the present disclosure can optionally include conventional food additives, such as any of, acidulants, additional thickeners, buffers or agents for pH adjustment, chelating agents, colorants, emulsifiers, excipients, flavor agents, minerals, osmotic agents, pharmaceutically acceptable carriers, preservatives, stabilizers, sugars, sweeteners, texturizers, or combinations thereof. The optional ingredients can be added in any suitable amount.

In summary, Applicant has surprisingly found that protein ingestion after consecutive resistance and endurance exercise selectively increased rates of myofibrillar, but not mitochondrial, protein synthesis in the early (e.g., 4 hours) recovery period. Applicant has also found that protein ingestion also attenuated post-exercise increases in genetic markers associated with muscle proteolysis. Given that endurance exercise interferes in strength/hypertrophy adaptation responses with concurrent training, the present findings suggest that protein intake can be beneficial following successive resistance and endurance exercise by promoting myofibrillar protein synthesis and decreasing ubiquitin ligase expression. Accordingly, post-exercise protein ingestion may ameliorate the potential “interference effect” of endurance exercise on muscle hypertrophy, and represents an important nutritional strategy for concurrent training.

The foregoing may be better understood by reference to the following Examples, which are presented for purposes of illustration and are not intended to limit the scope of the present disclosure.

### EXAMPLES

Applicant performed studies that demonstrate that the ingestion of a whey-protein supplement following consecutive resistance and endurance exercise (i.e., concurrent training) selectively increases rates of myofibrillar (i.e. contractile, but not mitochondrial,) protein synthesis in the early (e.g., 4 hour) recovery period following the training Protein ingestion also attenuated post-exercise increases in muscle breakdown. Given that endurance exercise can interfere with strength adaptations during concurrent training, these results can be used to communicate the importance of ingesting a high quality protein (e.g., whey) to ameliorate the potential “interference effect” of endurance exercise on muscle hypertrophy.

### METHODOLOGY AND TRIALS

The present experiments were performed at the Royal Melbourne Institute of Technology, Australia. Eight healthy male subjects (age 19.1±1.4 yr, body mass 78.1±15.6 kg, peak oxygen uptake (“VO₂ peak”) 46.7±4.4 mL·kg−1·min−1, leg extension one repetition maximum (“1-RM”) 130±14 kg; values are mean value±standard deviation] who had been participating in regular concurrent resistance and endurance training (˜3 times/week; >1 year) volunteered to participate in this study. The experimental procedures and possible risks associated with the study were explained to all subjects, who gave written informed consent before participation. The study was approved by the Human Research Ethics Committee of RMIT University.

Study Design

The study employed a randomized double-blind, cross-over design in which each subject completed two acute concurrent resistance and cycling exercise sessions with either post-exercise placebo (“PLA”) or protein (“PRO”) ingestion separated by a three week recovery period, during which time subjects maintained their habitual physical activity pattern.

Preliminary Testing

Peak Oxygen Uptake

Peak oxygen uptake was determined during an incremental test to volitional fatigue on a Lode cycle ergometer. In brief, subjects commenced cycling at a workload equivalent to 2 W/kg for 150 seconds. Thereafter, the workload was increased by 25 W every 150 seconds until volitional fatigue, defined as the inability to maintain a cadence >70 revolutions/minutes. Throughout the test, the subjects breathed through a mouthpiece attached to a metabolic cart to determine oxygen consumption.

Maximal Strength

Quadriceps strength was determined during a series of single repetitions on a plate-loaded leg extension machine until the maximum load lifted was established (1 RM). Repetitions were separated by a 3 minute recovery and were used to establish the maximum load/weight that could be moved through the full range of motion once, but not a second time. Exercise range of motion was 85° with leg extension endpoint set at −5° from full extension.

Diet/Exercise Control

Before an experimental trial subjects were instructed to refrain from exercise training and vigorous physical activity, and alcohol and caffeine consumption for a minimum of 48 hours. Subjects were provided with standardized pre-packed meals that consisted of 3 g carbohydrate/kg body mass, 0.5 g protein/kg body mass, and 0.3 g fat/kg body mass consumed as the final caloric intake the evening before reporting for an experimental trial.

Experimental Testing Session

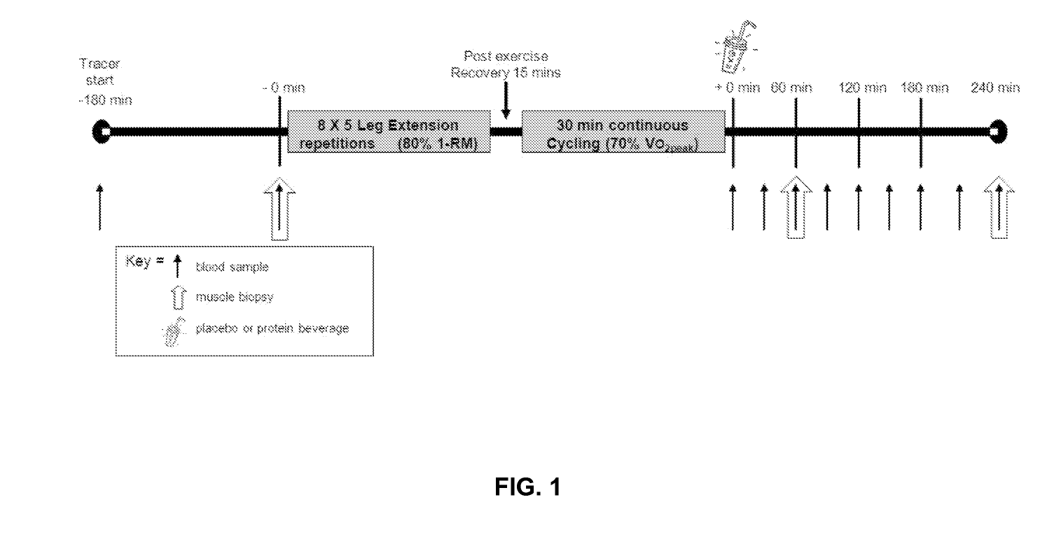

On the morning of an experimental trial, subjects reported to the laboratory after a ˜10 hour overnight fast. After resting in the supine position for ˜15 minutes, catheters were inserted into the anticubital vein of each arm and a baseline blood sample (˜3 mL) was taken (see, e.g., FIG. 1). A primed constant intravenous infusion (prime: 2 μmol·kg−1; infusion: 0.05 μmol·kg−1·min−1) of L-[ring-13C6] phenylalanine was then administered. Under local anaesthesia (2-3 mL of 1% Xylocalne) a resting biopsy was obtained 3 hours after commencement of the tracer infusion from the vastus lateralis using a 5-mm Bergstrom needle modified with suction. Subjects then completed the exercise intervention (described above). Immediately following the cessation of exercise, subjects ingested 500 mL of either a placebo (PLA: water, artificial sweetener) or protein beverage (PRO: 25 g whey protein). The protein beverage was enriched to 5% L-[ring-13C6] phenylalanine to prevent dilution of the steady-state isotope enrichment implemented by the constant infusion. Subjects rested throughout a 240 minute recovery period and additional muscle biopsies were taken 60 and 240 minutes post-exercise. Each muscle biopsy was taken from a separate site 2-3 cm distal from the right leg for the first trial and left leg for the second trial with all samples stored at −80° C. until subsequent analysis. Blood samples were collected in blood collection tubes (e.g., ethylenediaminetetraacetic acid (“EDTA”) tubes) at regular intervals during the post-exercise recovery period.

Resistance Exercise

After a standardized warm-up (2×5 repetitions at ˜50% and ˜60% 1 RM, respectively), subjects performed eight sets of five repetitions at ˜80% 1 RM. Each set was separated by a 3 minute recovery period during which time the subject remained seated on the leg extension machine. Contractions were performed at a set metronome cadence approximately equal to 30°/s and strong verbal encouragement was provided during each set. Subjects then rested for 15 minutes before beginning the cycling protocol.

Cycling Exercise

Subjects performed 30 minutes of continuous cycling at a power output that elicited ˜70% of individual VO₂ peak. Subjects were fan-cooled and allowed ad libitum access to water throughout the ride. Visual feedback for pedal frequency, power output, and elapsed time were provided to subjects.

Analytical Procedures

Blood Glucose and Plasma Insulin Concentration

Whole blood samples (5 mL) were immediately analyzed for glucose concentration using an automated glucose analyzer). Blood samples were then centrifuged at 1000 g at 4° C. for 15 minutes, with aliquots of plasma frozen in liquid N₂ and stored at −80° C. Plasma insulin concentration was then measured using a radioimmunoassay kit according to the manufacturer's protocol.

Plasma Amino Acids and Enrichment

Plasma amino acid concentrations were determined by high performance liquid chromatography (“HPLC”) from a modified protocol. Moore D R, Robinson M J, Fry J L, Tang J E, Glover E I, Wilkinson S B, Prior T, Tarnopolsky M A, and Phillips S M. 2009. Ingested protein dose response of muscle and albumin protein synthesis after resistance exercise in young men. The American Journal of Clinical Nutrition. 89: 161-168. Briefly, 100 μL of plasma was mixed with 500 μL of ice cold 0.6 M PCA and neutralized with 250 μL of 1.25 M potassium bicarbonate (“KHCO₃”). Samples were then subsequently derivatized for HPLC analysis.

Mitochondrial and Myofibrillar Protein Synthesis

A piece frozen of wet muscle (˜100 mg) was homogenized with a Dounce glass homogenizer on ice in an ice-cold homogenizing buffer (1M Sucrose, 1M Tris/HCl, 1M KCl, 0.5M EDTA) supplemented with a protease inhibitor and phosphatase cocktail tablet (e.g., PhosSTOP, Roche Applied Science, Mannhein, Germany) per 10 ml of buffer. The homogenate was transferred to an eppendorf tube and centrifuged to pellet a fraction enriched with myofibrillar proteins and collagen that was stored at −80° C. for subsequent extraction of the myofibrillar fraction (described below). The supernatant was transferred to another eppendorf tube and centrifuged to pellet the mitochondrial enriched protein fraction. The supernatant was placed in a separate eppendorf and stored at −80° C. for Western Blot analysis (described below). The mitochondrial enriched pellet was then washed, lyophilized and amino acids were liberated by adding 1.5 mL of 6M HCl and heating to 110° C. overnight. The myofibrillar pellet stored at −80° C. was washed twice with the homogenization buffer, centrifuged and supernatant was discarded. Myofibrillar proteins were solubilized in 0.3 M sodium hydroxide and precipitated with 1 M perchloric acid. Amino acids were then liberated from the myofibrillar enriched precipitate by adding 2.0 ml of 6 M HCl and heating to 110° C. overnight. Free amino acids from myofibrillar and mitochondrial enriched fractions were purified using cation-exchange chromatography and converted to their N-acetyl-n-propyl ester derivatives for analysis by gas chromatography combustion-isotope ratio mass spectrometry. Intracellular amino acids (“IC”) were extracted from a separate piece of wet muscle (˜20 mg) with ice-cold 0.6 M PCA. Muscle was homogenized and the free amino acids in the supernatant were purified by cation-exchange chromatography and converted to their heptafluorobutyric (“HFB”) derivatives before analysis by gas chromatography—mass spectrometry (“GC-MS”).

Calculations

The rate of mitochondrial and myofibrillar protein synthesis was calculated using the standard precursor-product method:

FSR(%·h−1)=[(E2b−E1b)/(EIC×t)]×100

where “E2b−E1b” represents the change in the bound protein enrichment between two biopsy samples, and “EIC” is the average enrichment of intracellular phenylalanine between the two biopsy samples and t is the time between two sequential biopsies.

Western Blots

The supernatant frozen at −80° C. from the previous mitochondrial enriched fraction extraction was used for determination of protein concentration using a BCA protein assay). The supernatant was subsequently resuspended in Laemelli sample buffer, separated by SDS-PAGE, transferred to polyvinylidine fluoride membranes and incubated with primary antibody (1:1,000) overnight at 4° C. on a shaker. Membranes were incubated with secondary antibody (1:2,000), and proteins were detected via enhanced chemiluminescence, and quantified by densitometry. All sample (40 μg) time points for each subject were run on the same gel. Polyclonal anti-phospho-AktSer473, -mTORSer2448, -Glycogen Synthase (“GS”) Ser641, -eEF2Thr56, and monoclonal anti-AMPKαThr172 and p70S6KThr389 were from Cell Signaling Technology. Data are expressed relative to α-tubublin in arbitrary units.

RNA Extraction and Quantification

Skeletal muscle tissue RNA extraction was performed on previously snap frozen samples with TRIzol according to the manufacturer's directions. Briefly, ˜20 mg of skeletal muscle was homogenized in TRIzol and chloroform added to form an aqueous RNA phase. This RNA phase was then precipitated by mixing with isopropanol alcohol and the resulting pellet was washed and re-dissolved in 50 μl of RNase-free water. Extracted RNA was quantified using a QUANT-iT analyser kit according to the manufactures directions. Quality of RNA was further determined on a NanoDrop 1000 spectrophotometer by measuring absorbance at 260 nm and 280 nm with a 260/280 ratio of ˜1.88 recorded for all samples. The RNA samples were diluted as appropriate to equalize concentrations, and stored at −80° C. for subsequent reverse transcription.

Reverse Transcription and Real-Time PCR

First-strand complementary DNA (“cDNA”) synthesis was performed using commercially available TaqMan Reverse Transcription Reagents in a final reaction volume of 20 μL. All RNA and negative control samples were reverse transcribed to cDNA in a single run from the same reverse transcription master mix. Serial dilutions of a template RNA was included to ensure efficiency of reverse transcription and for calculation of a standard curve for real-time quantitative polymerase chain reaction (“RT-PCR”). Quantification of mRNA (in duplicate) was performed on a 72-well centrifugal real-time cycler. Taqman-FAM-labeled primer/probes for MuRF-1, Atrogin, Myostatin, PGC-1α, Hexokinase and VEGF were used in a final reaction volume of 20 μL. PCR treatments were 2 minutes at 50° C. for UNG activation, 10 minutes at 95° C. then 40 cycles of 95° C. for 15 seconds and 60° C. for 60 seconds. Glyceraldehyde-3-phosphate dehydrogenase (“GAPDH”) was used as a housekeeping gene to normalize threshold cycle (“CT”) values. The relative amounts of mRNAs were calculated using the relative quantification (ΔΔCT) method.

Statistical Analysis

All data were analyzed by two-way ANOVA (two factor: time×treatment) with repeated measures and Student-Newman-Keuls post hoc analysis. Statistical significance was established when P<0.05. All data are expressed as arbitrary units±standard deviation.

Results

Plasma Insulin, Amino Acids and Blood Glucose

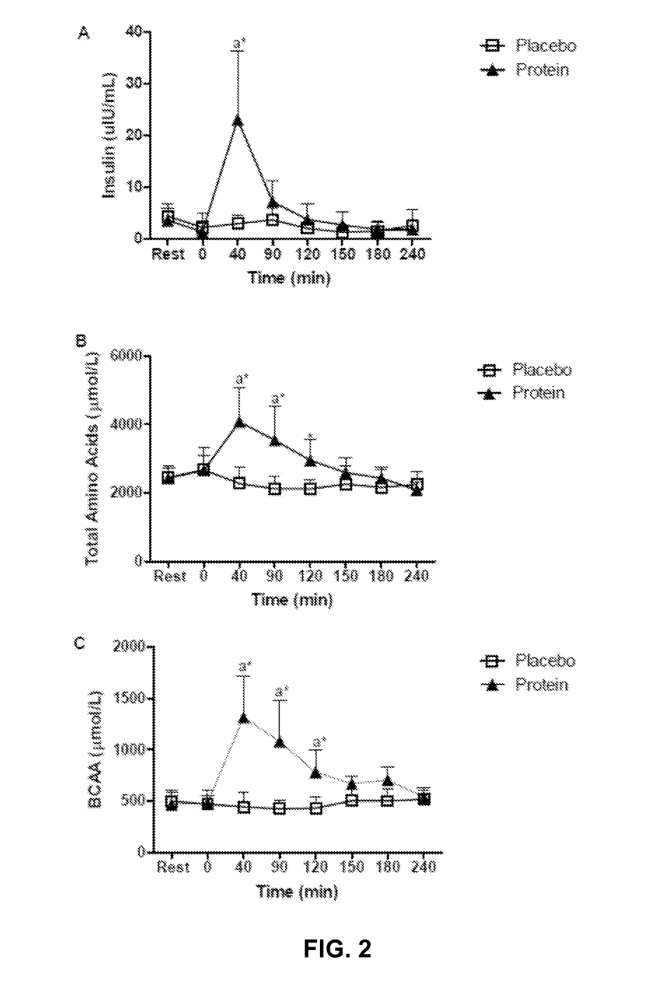

There were main effects for plasma insulin and total amino acid concentration with PRO but not PLA (P<0.001; see, e.g., FIGS. 2A and B). Peak plasma insulin (˜535%) and amino acid (˜70%) concentrations occurred 40 minutes post-exercise (P<0.001). The same effect was evident for branched-chain amino acids (BCAA) concentration (˜180%, P<0.001; see, e.g., FIG. 2C). Blood glucose was not different at any time in either treatment.

Plasma Tracer Enrichments

Plasma [ring 13C6] phenylalanine enrichment at rest, and 60, 120, 180 and 240 minutes post-exercise for PRO and PLA were 0.0688, 0.0557, 0.0679, 0.0673 and 0.0609, and 0.0617 0.0558, 0.0616, 0.0558, and 0.0606 tracer-to-tracee ratio: t·T−1, respectively. Linear regression analysis indicated that the slopes of the plasma enrichments were not significantly different from zero, showing isotopic plateau/steady-state.

Cell Signaling

Akt-mTOR-p70S6K-eEF2

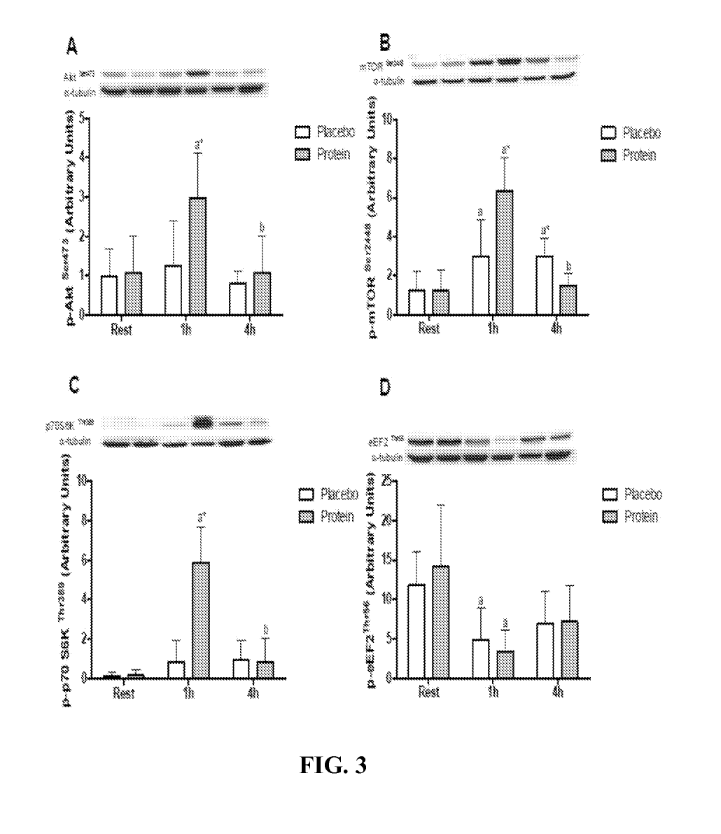

There were main effects for AktSer473 phosphorylation for time and treatment (P<0.05, see, e.g., FIG. 3A). AktSer473 phosphorylation increased above rest with PRO (˜175%; P<0.05) but not PLA 1 hour after exercise. This disparity in AktSer473 resulted in a significant difference between treatments at 1 hour (P<0.05). Phosphorylation in PRO then returned to resting levels 4 hours following recovery from exercise (P<0.05). There were main effects for time and treatment for mTORSer2448 phosphorylation (P<0.05, see, e.g., FIG. 3B). mTOR phosphorylation increased after PRO (˜400%, P<0.001) and PLA (˜100%, P<0.05) ingestion at 1 hour, and this increase was markedly higher with PRO (˜300%, P<0.001). mTORSer2448 phosphorylation remained elevated above rest 4 hours post-exercise with PLA only (˜130%, P<0.05), resulting in a significant disparity between treatments (P<0.05).

There were main effects for p70S6KThr389 phosphorylation for both time and treatment (P<0.05, see, e.g., FIG. 3C). p70S6KThr389 phosphorylation increased above rest with PRO (˜3000%; P<0.001) but not PLA 1 hour after exercise. This disparity in p70S6KThr389 resulted in a significant difference between treatments at 1 hour (P<0.05). Phosphorylation of p70S6K after PRO returned to resting levels after 4 hours of recovery from exercise (P<0.001). There were main effects for eEF2Thr56 phosphorylation for time in both treatments (P<0.05, see, e.g., FIG. 3D). One hour post-exercise, phosphorylation of eEF2 decreased ˜60% (P<0.05) with PLA and ˜75% (P<0.05) with PRO and remained at this level for the duration of the recovery (4 hours).

AMPK-GS

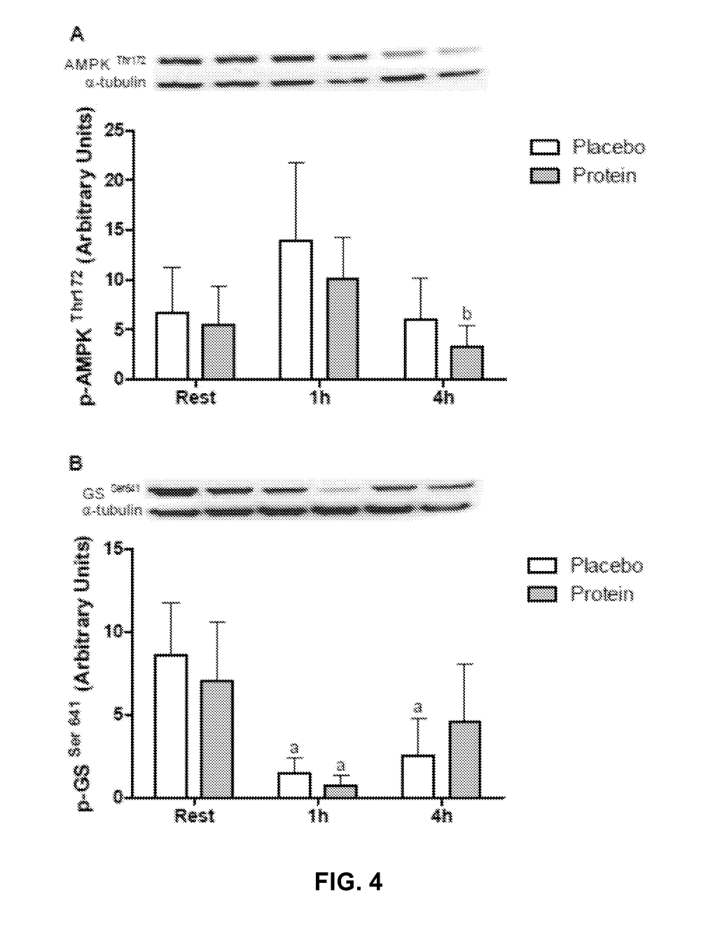

There were main effects for both time and treatment for AMPKThr172 phosphorylation (P<0.05, see, e.g., FIG. 4A). AMPKThr172 phosphorylation decreased from 1 hour to 4 hours post-exercise after PRO only (˜70%, P<0.05) and was higher in PLA, however post hoc analysis failed to show any differences for any individual time point. There were main effects for time for GSSer64′ phosphorylation (P<0.05, see, e.g., FIG. 4B). GSSer641 phosphorylation was lower from rest at 1 hour (˜80%, P<0.05) and 4 hours (˜70%, P<0.05) post-exercise with PLA. GS phosphorylation similarly decreased at 1 hour with PRO compared to rest (˜90%, P<0.05) but was not different at 4 hours.

mRNA Expression

MuRF1-Atrogin-1-Myostatin

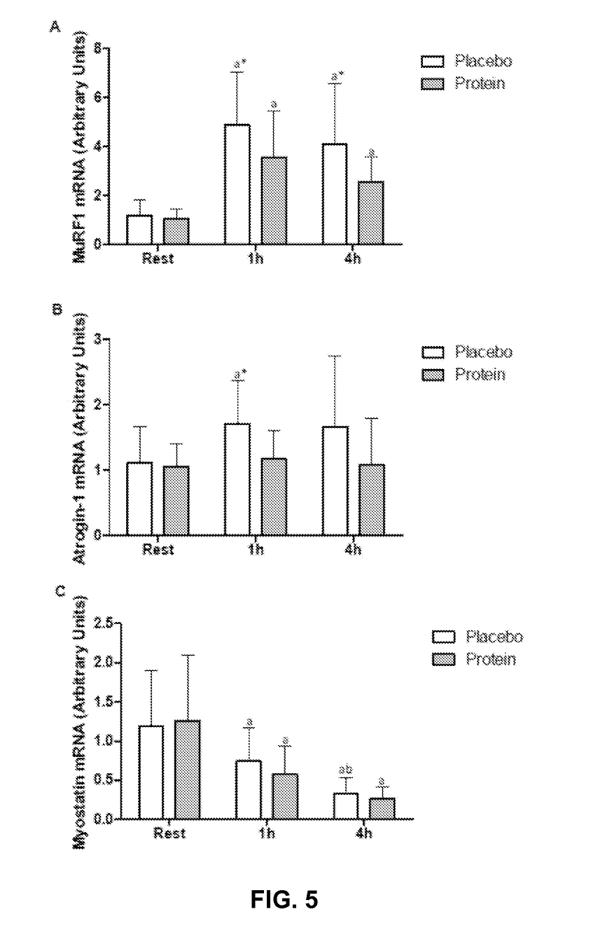

There were main effects for time and treatment for MuRF1 mRNA abundance (P<0.05, see, e.g., FIG. 5A). MuRF1 increased significantly above resting levels at 1 hour (˜315% vs. ˜230%, P<0.001) and 4 hours (˜250% vs. ˜140%, P<0.05) post-exercise after both PLA and PRO, respectively. MuRF1 was higher in PLA compared to PRO at both post-exercise time points (1 hour: 78%, 4 hours: 105%, P<0.05). Atrogin-1 mRNA expression increased above rest only with PLA 1 hour post-exercise (˜50%, P<0.05; see, e.g., FIG. 5B). The disparity in Atrogin-1 mRNA at 1 hour resulted in a significant difference between treatments (P<0.05). There was a main effect of time for myostatin mRNA abundance (P<0.05, see, e.g., FIG. 5C). Myostatin decreased from rest at 1 hour (˜40% vs. ˜55%, P<0.05) and 4 hours (˜70% vs. ˜80%, P<0.001) after both PLA and PRO, respectively. Myostatin mRNA at 1 hour was different from 4 hours after PLA (˜120%, P<0.05).

PGC-1α-Hexokinase-VEGF

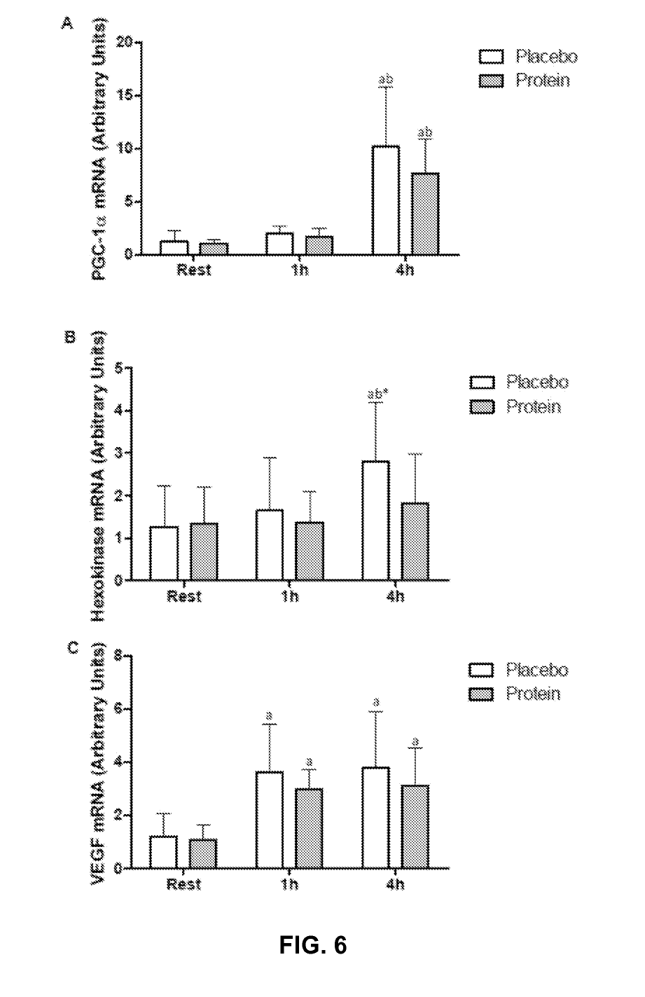

There were main effects for PGC-1α mRNA abundance for time (P<0.05, see, e.g., FIG. 6A). PGC-1α expression increased above resting and 1 hour levels following 4 hours post-exercise recovery in PLA (˜730%, P<0.001) and PRO (˜620%, P<0.001). There were main effects for time and treatment for hexokinase mRNA expression (P<0.05, see, e.g., FIG. 6B). Hexokinase increased above rest at 4 hours in PLA only (˜120%, P<0.05) whereas in PRO there were no changes. This disparity resulted in a significant difference between treatments at 4 hours (P<0.05). VEGF mRNA expression increased above rest at both 1 hour (˜200%, P<0.001) and 4 hours (˜210%, P<0.001) with PLA (see, e.g., FIG. 6C). Likewise, VEGF also increased with PRO at 1 hour (˜170%, p<0.05) and 4 hours (˜180; P<0.05). There were no differences between treatments at any post-exercise time point.

Muscle Protein Synthesis

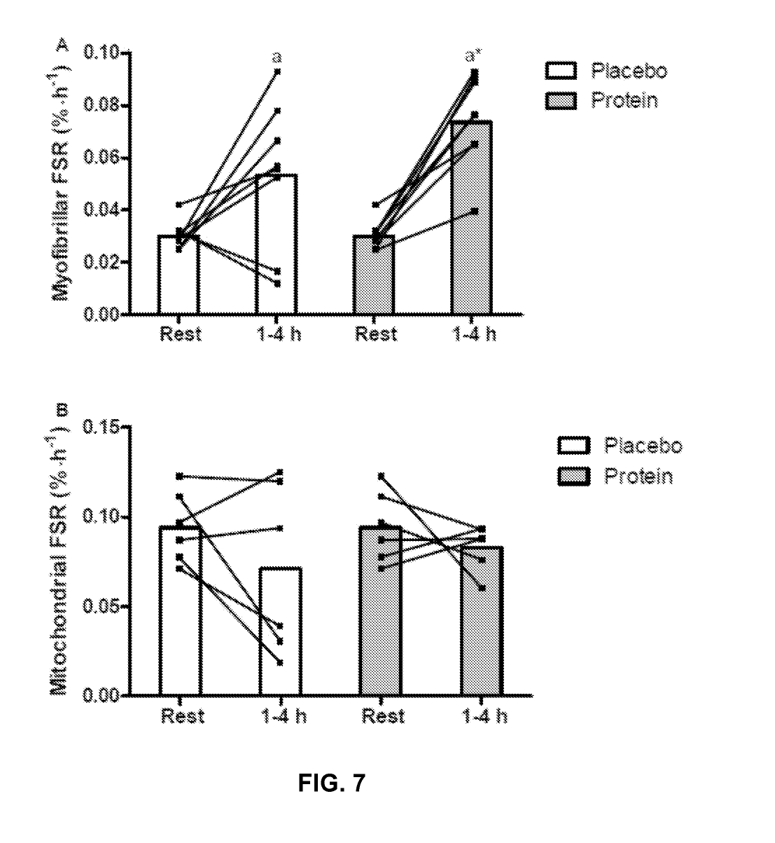

Rates of myofibrillar protein synthesis increased above rest between 1 hour and 4 hours post-exercise after both PLA (˜75%, P<0.05) and PRO (˜145%, P<0.001) (see, e.g., FIG. 7A). This post-exercise increase in the rate of myofibrillar synthesis was greater with PRO compared to PLA (P<0.05). Rates of mitochondrial protein synthesis (n=6) were unchanged during the acute post-exercise period and there were no differences in post-exercise fractional synthesis rates between treatments (see, e.g., FIG. 7B).

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A method for enhancing muscle protein synthesis following physical exertion comprising administering to an individual a composition comprising from about 15 g to about 35 g protein from about 0 to about 30 minutes after concurrent training.

2. The method according to claim 1, wherein the protein is selected from the group consisting of dairy based proteins, plant based proteins, animal based proteins, artificial proteins, and combinations thereof.

3. The method according to claim 1, the composition further comprising essential amino acids selected from the group consisting of phenylalanine, valine, threonine, tryptophan, isoleucine, methionine, leucine, lysine, histidine, and combinations thereof.

4. The method according to claim 1, wherein the composition is enriched with L-[ring-13C6] phenylalanine in an amount up to about 10% by weight of the composition.

5. The method according to claim 1, the composition further comprising at least one of:
a) a prebiotic selected from the group consisting of acacia gum, alpha glucan, arabinogalactans, beta glucan, dextrans, fructooligosaccharides, fucosyllactose, galactooligosaccharides, galactomannans, gentiooligosaccharides, glucooligosaccharides, guar gum, inulin, isomaltooligosaccharides, lactoneotetraose, lactosucrose, lactulose, levan, maltodextrins, milk oligosaccharides, partially hydrolyzed guar gum, pecticoligosaccharides, resistant starches, retrograded starch, sialooligosaccharides, sialyllactose, soyoligosaccharides, sugar alcohols, xylooligosaccharides, their hydrolysates, and combinations thereof;
b) a probiotic selected from the group consisting of probiotics include Aerococcus, Aspergillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, and combinations thereof;
c) a phytonutrient selected from the group consisting of flavanoids, allied phenolic compounds, polyphenolic compounds, terpenoids, alkaloids, sulphur-containing compounds, and combinations thereof;
d) an antioxidant selected from the group consisting of astaxanthin, carotenoids, coenzyme Q10 (“CoQ10”), flavonoids, glutathione, Goji (wolfberry), hesperidin, lactowolfberry, lignan, lutein, lycopene, polyphenols, selenium, vitamin A, vitamin C, vitamin E, zeaxanthin, and combinations thereof;
e) a vitamin, wherein the vitamin is selected from the group consisting of vitamin A, Vitamin B1 (thiamine), Vitamin B2 (riboflavin), Vitamin B3 (niacin or niacinamide), Vitamin B5 (pantothenic acid), Vitamin B6 (pyridoxine, pyridoxal, or pyridoxamine, or pyridoxine hydrochloride), Vitamin B7 (biotin), Vitamin B9 (folic acid), and Vitamin B12 (various cobalamins; commonly cyanocobalamin in vitamin supplements), vitamin C, vitamin D, vitamin E, vitamin K, K1 and K2 (i.e., MK-4, MK-7), folic acid, biotin, and combinations thereof;
f) a mineral, wherein the mineral is selected from the group consisting of boron, calcium, chromium, copper, iodine, iron, magnesium, manganese, molybdenum, nickel, phosphorus, potassium, selenium, silicon, tin, vanadium, zinc, and combinations thereof; or
g) combinations thereof.

6. The method according to claim 1, wherein the protein synthesis enhanced is myofibrillar protein synthesis and/or mitochondrial protein synthesis.

7. The method according to claim 1, wherein a serving size of the composition is about 500 mL.

8. A program for enhancing muscle adaptation resulting from concurrent training comprising providing nutrition and guidance on training to an athlete, the program comprising:
a. providing a composition comprising from about 15 g to about 35 g protein; and
b. providing guidelines for consumption comprising a recommendation of the amount of the composition to consume following concurrent training.

9. The program according to claim 8, wherein the program comprises recommendations to undertake concurrent training at 1 to 3 times per week, for 1 to 6 weeks; and
the composition is administered from about 0 to about 30 minutes after concurrent training.

10. The program according to claim 8, the composition further comprising essential amino acids selected from the group consisting of phenylalanine, valine, threonine, tryptophan, isoleucine, methionine, leucine, lysine, histidine, and combinations thereof.

11. The program according to claim 8, wherein the composition is enriched with L-[ring-13C6] phenylalanine in an amount up to about 10% by weight of the composition.

12. The program according to claim 8, the composition further comprising at least one of:
a) a prebiotic selected from the group consisting of acacia gum, alpha glucan, arabinogalactans, beta glucan, dextrans, fructooligosaccharides, fucosyllactose, galactooligosaccharides, galactomannans, gentiooligosaccharides, glucooligosaccharides, guar gum, inulin, isomaltooligosaccharides, lactoneotetraose, lactosucrose, lactulose, levan, maltodextrins, milk oligosaccharides, partially hydrolyzed guar gum, pecticoligosaccharides, resistant starches, retrograded starch, sialooligosaccharides, sialyllactose, soyoligosaccharides, sugar alcohols, xylooligosaccharides, their hydrolysates, and combinations thereof;
b) a probiotic selected from the group consisting of probiotics include Aerococcus, Aspergillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, and combinations thereof;
c) a phytonutrient selected from the group consisting of flavanoids, allied phenolic compounds, polyphenolic compounds, terpenoids, alkaloids, sulphur-containing compounds, and combinations thereof;
d) an antioxidant selected from the group consisting of astaxanthin, carotenoids, coenzyme Q10 (“CoQ10”), flavonoids, glutathione, Goji (wolfberry), hesperidin, lactowolfberry, lignan, lutein, lycopene, polyphenols, selenium, vitamin A, vitamin C, vitamin E, zeaxanthin, and combinations thereof;
e) a vitamin, wherein the vitamin is selected from the group consisting of vitamin A, Vitamin B1 (thiamine), Vitamin B2 (riboflavin), Vitamin B3 (niacin or niacinamide), Vitamin B5 (pantothenic acid), Vitamin B6 (pyridoxine, pyridoxal, or pyridoxamine, or pyridoxine hydrochloride), Vitamin B7 (biotin), Vitamin B9 (folic acid), and Vitamin B12 (various cobalamins; commonly cyanocobalamin in vitamin supplements), vitamin C, vitamin D, vitamin E, vitamin K, K1 and K2 (i.e., MK-4, MK-7), folic acid, biotin, and combinations thereof;
f) a mineral, wherein the mineral is selected from the group consisting of boron, calcium, chromium, copper, iodine, iron, magnesium, manganese, molybdenum, nickel, phosphorus, potassium, selenium, silicon, tin, vanadium, zinc, and combinations thereof; or
g) combinations thereof.

13. The program according to claim 8, wherein the program is for enhancing protein synthesis resulting from concurrent training.

14. The program according to claim 13, wherein the protein synthesis enhanced is myofibrillar protein synthesis and/or mitochondrial protein synthesis.

15. A nutritional kit for enhancing muscle adaptation comprising a plurality of compositions comprising from about 15 g to about 35 g protein and guidelines recommending that an athlete consume the composition from about 0 to about 30 minutes after concurrent training.

16. The nutritional kit according to claim 15, the composition further comprising essential amino acids selected from the group consisting of phenylalanine, valine, threonine, tryptophan, isoleucine, methionine, leucine, lysine, histidine, and combinations thereof.

17. The nutritional kit according to claim 15, wherein the composition is enriched with L-[ring-13C6] phenylalanine in an amount up to about 10% by weight of the composition.

18. The nutritional kit according to claim 15, the composition further comprising at least one of:
a) a prebiotic selected from the group consisting of acacia gum, alpha glucan, arabinogalactans, beta glucan, dextrans, fructooligosaccharides, fucosyllactose, galactooligosaccharides, galactomannans, gentiooligosaccharides, glucooligosaccharides, guar gum, inulin, isomaltooligosaccharides, lactoneotetraose, lactosucrose, lactulose, levan, maltodextrins, milk oligosaccharides, partially hydrolyzed guar gum, pecticoligosaccharides, resistant starches, retrograded starch, sialooligosaccharides, sialyllactose, soyoligosaccharides, sugar alcohols, xylooligosaccharides, their hydrolysates, and combinations thereof;
b) a probiotic selected from the group consisting of probiotics include Aerococcus, Aspergillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, and combinations thereof;
c) a phytonutrient selected from the group consisting of flavanoids, allied phenolic compounds, polyphenolic compounds, terpenoids, alkaloids, sulphur-containing compounds, and combinations thereof;
d) an antioxidant selected from the group consisting of astaxanthin, carotenoids, coenzyme Q10 (“CoQ10”), flavonoids, glutathione, Goji (wolfberry), hesperidin, lactowolfberry, lignan, lutein, lycopene, polyphenols, selenium, vitamin A, vitamin C, vitamin E, zeaxanthin, and combinations thereof;
e) a vitamin, wherein the vitamin is selected from the group consisting of vitamin A, Vitamin B1 (thiamine), Vitamin B2 (riboflavin), Vitamin B3 (niacin or niacinamide), Vitamin B5 (pantothenic acid), Vitamin B6 (pyridoxine, pyridoxal, or pyridoxamine, or pyridoxine hydrochloride), Vitamin B7 (biotin), Vitamin B9 (folic acid), and Vitamin B12 (various cobalamins; commonly cyanocobalamin in vitamin supplements), vitamin C, vitamin D, vitamin E, vitamin K, K1 and K2 (i.e., MK-4, MK-7), folic acid, biotin, and combinations thereof;
f) a mineral, wherein the mineral is selected from the group consisting of boron, calcium, chromium, copper, iodine, iron, magnesium, manganese, molybdenum, nickel, phosphorus, potassium, selenium, silicon, tin, vanadium, zinc, and combinations thereof; or
g) combinations thereof.

19. The nutritional kit according to claim 15, wherein the program is for enhancing protein synthesis resulting from concurrent training.

20. The nutritional kit according to claim 19, wherein the protein synthesis enhanced is myofibrillar protein synthesis and/or mitochondrial protein synthesis.
